(19) United States
(12) Patent Application Publication
(10) Pub. No.: US 2010/0112722 A1
(43) Pub. Date: May 6, 2010

(54) Immunoassays and Characterization of Biomolecular Interactions Using Self-Assembled Monolayers

(76) Inventors: Milan Mrksich, Hinsdale, IL (US); Violeta L. Marin, Chicago, IL (US); Steven M. Patrie, Dallas, TX (US)

Correspondence Address: BRINKS HOFER GILSON & LIONE, P.O. BOX 10395, CHICAGO, IL, 60610, US

(21) Appl. No.: 12/565,575

(22) Filed: Sep. 23, 2009

Related U.S. Application Data
(63) Continuation of application No. PCT/US2008/058267, filed on Mar. 26, 2008.
(60) Provisional application No. 60/920,009, filed on Mar. 26, 2007.
(60) Provisional application No. 60/928,496, filed on May 9, 2007.

(51) Int. Cl. G01N 33/53

(52) U.S. Cl. 436/501

(57) ABSTRACT

The present disclosure relates to methods of characterizing protein-protein interaction or conducting immunoassays in a biological sample using self-assembled monolayers (SAMs) and matrix-assisted laser desorption/ionization time of flight mass spectrometry (SAMDI). The biological sample may be obtained from a living subject, such as a human or animal clinical sample containing multiple unknown proteins and/or antigens. Label-free methods of identifying protein-protein interactions, antigen-antibody binding and/or diagnosing a medical condition based on analysis of a biological sample using SAMDI are also provided, as well as biochips comprising surface bound proteins and/or antibodies and methods of making these biochips. The methods and biochips are useful, for example, for identifying protein-protein binding interactions and/or conducting immunoassays in samples such as humoral fluids or other clinical samples, cell lysates, tissue lysates, tumor lysates, and samples obtained, isolated or derived from animals or plants.

### RELATED APPLICATIONS

This application is a continuation of PCT International Serial Number PCT/US2008/058267, filed Mar. 26, 2008, which designates the U.S. and was published in English, and which claims priority to U.S. Provisional Patent Application Ser. No. 60/920,009, entitled “CHARACTERIZATION OF BIOMOLECULAR INTERACTIONS USING SELF-ASSEMBLED MONOLAYERS,” filed Mar. 26, 2007 and U.S. Provisional Patent Application Ser. No. 60/928,496, entitled “CHARACTERIZATION OF BIOMOLECULAR INTERACTIONS USING SELF-ASSEMBLED MONOLAYERS,” filed May 9, 2007, both of which are incorporated herein by reference in their entirety.

### STATEMENT OF FEDERAL RESEARCH FUNDING

This invention was made with government support under CCNE NIH Award Number U54CA119341 awarded by the National Institutes of Health (NIH) and under MRSEC Grant Number NSF DMR-0213745 awarded by the National Science Foundation (NSF). The government has certain rights in this application.

### TECHNICAL FIELD

The present disclosure pertains to the characterization of protein interactions and antibody-antigen binding by analyzing binding to self-assembled monolayers. In particular, laser desorption ionization mass spectrometry may be used to analyze the biomolecular binding of analytes to self-assembled monolayers presenting selected molecular species, such as proteins, antibodies or antigens.

### BACKGROUND

The genomic era has introduced high throughput technologies for rapidly sequencing whole genomes and in turn has created an urgent need for technologies that analyze protein function. Indeed, the current capability to generate sequence of entire genomes far outpaces the development of high confidence methods for assignment of protein function. It is widely appreciated that proteins with high similarity in sequence or structure can carry out different functions and that many proteins have multiple functions. Any understanding of the roles and functions of proteins must include an experimental determination of the interactions that proteins participate in. For a set of n proteins, there are of order n²/2 possible interactions.

Various methods may be used for high throughput mapping of protein interactions. These existing methods include the yeast two-hybrid screen, which fuses proteins to transcription factor domains and relies on the reconstitution of transcriptional complexes when a ‘bait’ and ‘prey’ protein interact. Another cell-based method relies on immobilizing a bait protein to a solid phase and using affinity methods to isolate interacting partners from cell lysates, followed by mass spectrometry to identify the prey proteins. These, and related cell-based methods, have the advantages that they do not require high throughput cloning, expression, and purification of proteins for in vitro studies and therefore can be readily adapted to survey a large number of potential interactions. But, it can be difficult to unambiguously test each possible interaction in the background of many possible interactions. For example, a given pair of proteins may interact efficiently but the interaction is not observed if one of the proteins is interacting with another partner that is present at higher concentration (or has higher affinity) or that is localized in the same cellular compartment as the bait protein. These limitations—which may be particularly confounding for identifying multiple roles of a protein—can be circumvented by testing interactions of defined proteins in the absence of possible competing partners—that is, in performing a true one-on-one mapping of protein interactions.

Protein arrays provide an opportunity to achieve a broad scale one-on-one mapping of interactions within a large set of proteins. By applying a single protein in solution to an array of hundreds of proteins, for example, the ability to test each interaction in the absence of competing equilibria is straightforward. Label-dependent methods are often required to assay the interactions. The need for a label can add substantial time and cost to the preparation of reagents and can additionally compromise the activities of the interacting partners. Most importantly, the use of a label is not compatible with all types of assays and often prevents the identification of wholly unanticipated biochemical activities. There is a need for techniques that permit identification and characterization of these protein-protein interactions in the absence of a label.

Immunosensors hold a special importance in both basic research and clinical diagnostics. These assays use capture agents (either antibodies or antigens) that are immobilized on a solid phase to determine the concentration of a corresponding analyte in a biological sample. The generality of immunosensors stems from the availability of high affinity and selective antibodies to a broad range of analytes and to common “label-based” formats (using chromogenic, radioactive, or fluorescent reagents) for the detection of analytes. Virtually all assays that use biochip formats require the use of labels to identify enzymatic activities. The labels include fluorescent reporter groups (common in protease assays), isotopic labels (common in group transfer reactions) and antibodies. These labels have several drawbacks: fluorescent reporter systems often require laborious development (FRET, HTRF); antibodies selective for a particular modification are not always available (anti-phosphoserine/threonine antibodies are not general); and radioactive labels are undesirable and expensive for disposal. For applications that seek to identify interactions among hundreds of proteins, the cost and time required to label each protein (as well as the concern that this labeling strategy will, in many cases, compromise the activities of the proteins) is a great concern. Recently, increasing emphasis has been placed on the development of “label-free” detection technologies which hold much promise for diagnostic immunoassays. By eliminating the need for labeled reagents these label-free assays offer prospects of reduced time involved for assay development, simplified protocols, lower cost, and realization of immunosensors that perform multiple assays with a common sample.

Mass spectrometry (MS) is an important technique for characterizing the structures of surfaces and has several characteristics that are valuable in bioanalytical applications. In biochip and microarray applications, for example, MS offers the significant advantage that it does not require analytes to be labeled—either by direct attachment of fluorescent and radioactive labels or by binding of antibodies—and therefore offers greater flexibility in experiments. Yet, MS remains a secondary option to the use of fluorescence and radioactivity for characterizing biochips, in part because many early studies have used home-built instrumentation and sophisticated protocols were required for data analysis.

Matrix-assisted laser desorption/ionization and time of flight mass spectrometry (MALDI-TOF MS), when combined with self-assembled monolayers (SAMs) that are tailored for biological applications, is well suited for characterizing biological activities as illustrated by the following examples that characterize the immobilization of ligands, the selective binding of proteins, and the enzymatic modification of immobilized molecules. MALDI-TOF has been used for many years to identify peptides, proteins, carbohydrates and nucleic acids. In practice, aqueous samples are mixed with low molecular weight matrix molecules and dried on a metallic substrate prior to the MS analysis. Although MALDI MS is superior to other MS methods for analyzing complex biological mixtures comprising an endogenous material from a living subject, the presence of many components still leads to complicated spectra, which requires sophisticated analysis to identify specific analytes. Biochip applications, which rely on specific interactions of soluble and immobilized biomolecules, can avoid this limitation since only active components are retained on the substrate prior to MS analysis. Examples of biochip applications of MALDI technology include U.S. Pat. No. 7,172,905 entitled “Polypeptide immobilization” and filed Aug. 7, 2001; U.S. patent application Ser. No. 11/645,095 published as US 2008-0064606 A1 entitled “Polypeptide immobilization” and filed Dec. 21, 2006; and PCT patent application PCT/US03/21224, entitled “Characterization of Biochips Containing Self-Assembled Monolayers”, published as WO 2004/005918 and filed Jul. 7, 2003.

SAMs are well-suited for analysis by MALDI-TOF MS. This technique, termed self-assembled monolayers for matrix assisted laser desorption ionization mass spectrometry (SAMDI), provides the masses of substituted alkanethiols—permitting enzyme activity assays on immobilized peptides, carbohydrates, and small molecules—and can observe proteins bound to the monolayer with masses approaching 100 kD. SAMDI is well suited to the analysis of low molecular weight species, including peptides, carbohydrates and organic molecules. Indeed, SAMDI has been used to perform a range of enzyme activity assays—including kinase, protease, methyltransferase and glycosyltransferase activities—and has been applied to screening small molecule libraries to identify antagonists of enzyme activities.

Biochip technology based on self-assembled monolayers of alkanethiolates on gold may be applied to a broad range of enzyme assays—including those of kinase, phosphatase, glycosyltransferase, methyltransferase, deacetylation, protease and other activities—and to high throughput chemical screens. Key to the success of this method is the use of a surface chemistry that provides several functions. First, the structural integrity and synthetic flexibility of the monolayers are important for displaying immobilized biological reagents in regular environments, ensuring a uniform (and therefore, quantitative) activity of these moieties. Second, the use of monolayers that present oligo(ethylene glycol) groups are effective at preventing non-specific protein adsorption (and therefore, background signals). Finally, a portfolio of selective immobilization chemistries serves to control the densities of the immobilized proteins and in turn give uniform densities of distinct species across the entire array. Self assembled monolayers that present oligo(ethylene glycol) groups are highly effective at preventing the non-specific adsorption of protein. These surfaces maintain this property in complex solutions, including serum-containing cell culture media. The monolayers are structurally well-defined and can be synthetically modified to tailor the interfacial properties. Monolayers are compatible with multiple analytical methods used in characterizing biochips, including surface plasmon resonance spectroscopy, fluorescence imaging, radioisotope detection, and mass spectrometry. A full portfolio of immobilization chemistries that can be used to tether ligands to monolayers, and that provide for a defined orientation of the ligands and a rigorous control over the density, is available.

Mrksich et al. have developed SAM-based biochip surface chemistries that can be applied to a broad range of protein binding and enzyme activity assays that are compatible with several analytical formats, including SPR, radioactivity, fluorescence and mass spectrometry. The approach uses self-assembled monolayers of alkanethiolates on gold that are functionalized with oligo(ethylene glycol) groups and maleimide groups. The former are important for reducing the non-specific adsorption of proteins and the latter can be used to immobilize biologically active motifs or functional groups used for subsequent immobilization. An initial application of these methods to immunoassays using Surface Plasmon Resonance (SPR) detection described the preparation of fusion proteins containing antibody variable (Fv) domains fused to cutinase, a protein that irreversibly binds a phosphonate ligand (Kwon, Y.; Han, Z.; Karatan, E.; Mrksich, M.; Kay, B. K. Anal Chem 2004, 76, 5713-5720).

Significantly, the successful extension of SAMDI from detecting low molecular weight species to large molecular weight proteins, and from analyzing solutions of defined and relatively simple composition to complex samples derived from endogenous bodily fluids, has not been previously demonstrated. A first report showed that SAMDI could observe molecular ions corresponding to intact proteins with masses up to approximately 50 kDa (Yeo, W. S.; Min, D. H.; Hsieh, R. W.; Greene, G. L.; Mrksich, M., Angew Chem Int Ed Engl 2005, 44, 5480-5483). That work used surfaces presenting glutathione ligands to immobilize GST-fusion proteins, with the limitation that the rate constant for dissociation of the GST-glutathione interaction limited the stability of the biochip in diagnostic applications. Despite the benefits that protein-based mass spectrometry methods such as MALDI offer in clinical diagnostic assays, these methods are still not employed to analyze complex clinical samples. One reason for this slow acceptance stems from the usual barrier to the introduction of novel methods, but other factors include the need for technicians with specialized training and the need for methods that simplify the preparation, isolation and enrichment of the analytes (Verrills, N. M. Clin Biochem Rev 2006, 27, 99-116). Standardization of analytical methods has partly been addressed by the commercial mass spectrometer. Advances in biochip technology have provided a means for the enrichment of analytes from complex samples. For example, proteins may be partitioned onto a solid support through non-specific interactions with chromatographic resins arrayed onto the surface. In this way, analytes in a complex sample can be enriched through their preferential interaction with a solid phase resin. However, using these methods, analytes cannot be isolated from the sample as is done with immunoassays. This lack of specificity of the plate for an analyte, and the lack of standardized pre-analytical procedures for samples give rise to peak intensity CVs of 10-40%, and limited dynamic range for protein detection in complex mixtures (Albrethsen, J.; Bogebo, R.; Olsen, J.; Raskov, H.; Gammeltoft, S. Clin Chem Lab Med 2006, 44, 1243-1252). Strategies, such as the SAMDI immunoassay, which combine well defined surface chemistries for the selective and reproducible localization of analytes with mass spectrometry offers an alternative methodology to address many of the issues associated with standardized clinical diagnostics.

However, there remains a need for methods and devices for measuring protein-protein interactions and/or providing immunoassays in complex samples containing various unknown large proteins or molecules, such as body fluids. In particular, analytical methods for identifying protein-protein interactions and/or antibody-antigen interactions in an endogenous sample from a living subject are needed. For example, there is a need for techniques and devices for detecting these biomolecular interactions in a clinical sample using SAMDI techniques. In addition, there is a need for methods and devices that permit measurement and characterization of molecular interactions, such as measuring a binding interaction between specific proteins or conducting an immunoassay, without requiring conventional labeling techniques.

### SUMMARY

This disclosure describes methods to identify biomolecular interactions, such as protein-protein interactions and antigen-antibody binding, using self-assembled monolayer substrates (such as biochips). Demonstrated herein are applications of the SAMDI-based approach to biomolecular interactions including two exemplary applications of SAMDI to: (1) identify and characterize protein-protein interaction and (2) perform immunoassays for diagnostic applications and label-free detection of protein markers from complex samples, such as humoral fluids or other clinical samples, cell lysates, tissue lysates, tumor lysates, and samples obtained, isolated or derived from animals or plants.

In a first embodiment, methods for characterizing interaction between two biomolecules, which may be a first protein and a second protein, using a self-assembled monolayer (SAM) are provided. One or more SAM's may be adapted to selectively bind a first protein comprising a capture moiety, such as a capture tag or capture ligand, in the presence of a second protein without the capture tag, the method comprising the steps of: (a) contacting a first protein including the capture tag and a second protein free of the capture tag with the SAM in a manner effective to bind the capture tag to the SAM; (b) ionizing and ablating the SAM, the first protein bound to the SAM and any of the second protein bound to the first protein bound to the SAM and obtaining a mass spectrum including at least one mass-to-charge peak characteristic of the first protein bound to the SAM; and (c) analyzing the mass spectrum to determine if the second protein interacted with the first protein bound to the SAM. In a first aspect of the first embodiment, the first protein may be contacted with the SAM in a manner effective to bind at least a portion of the first protein comprising the capture tag to the SAM followed by contacting the second protein free of the capture tag with the SAM in a manner effective to bind the second protein to a portion of the first protein bound to the SAM. In a second aspect of the first embodiment, the first protein and the second protein are mixed together in a protein solution, and the protein solution is contacted with the SAM in a manner effective to permit at least a portion of the first protein to bind to the SAM and at least a portion of the second protein to bind to the portion of the first protein bound to the SAM. The mass spectrum typically includes a first peak characteristic of the first protein including the capture tag and at least one additional peak characteristic of the binding of the second protein to the first protein. The interaction of the first protein and the second protein is preferably analyzed on a biochip using self-assembled monolayers for matrix assisted laser desorption ionization mass spectrometry (SAMDI), in a manner that does not require detecting a fluorescent reporter group or a heterologous tag that is adapted to bind a fluorescent reporter.

In a second embodiment, methods for performing immunoassays using antibodies attached to SAMs in combination with self-assembled monolayer laser desorption/ionization time of flight mass spectrometry (SAMDI) mass spectrometry (MS) analysis are provided. Preferred methods include the steps of immobilizing antibodies on a SAM and performing immunoassays with SAMDI to analyze the binding of analytes, such as antigens, to the antibodies bound to the SAM. Antibodies or antigens may be immobilized to the SAM by contacting a SAM comprising a receptor protein with the antibody in a manner effective to bind the antibody to the receptor protein. The receptor protein may be any suitable Fc binding protein, such as Protein A or Protein G, selected to bind the to the heavy chain constant region of the antibody (Fc). Optionally, recombinant Protein G (or Protein A) containing an oligohistidine tag may be immobilized to the SAM and then used to capture an IgG antibody. The specific interactions that mediate the immobilization enforce a defined orientation of the antibody on the surface while not interfering with the antigen binding region on the antibody and therefore are expected to optimize the activity of the immobilized antibody. Further, this strategy offers control over the density of antibody, which can be particularly relevant to reducing the steric interactions of large antigens at the surface. The SAM preferably comprises an alkanethiol portion and an oligo(ethylene glycol) portion to reduce non-specific binding of proteins to the surface. The SAM comprising the bound antibody can be contacted with a sample in a manner effective to bind an antigen in the sample to the bound antibody. Matrix-assisted laser desorption ionization mass spectroscopy (such as SAMDI) of the SAM and bound antibody can provide a mass spectrum with mass-to-charge peaks indicative of the bound antibody, the SAM molecule and the presence of antigen bound to the bound antibody. The mass spectrum may be analyzed to determine if an antigen in the sample bound to the bound antibody. Optionally, the SAM may include multiple bound antibodies, and the resulting mass spectrum can indicate the presence of two or more antigens from the sample bound to the bound antibodies. Accordingly, the methods are useful as an immunoassay method for detecting one or more antigens in the sample.

In one aspect of the second embodiment, immunoassay methods may be used to determine the presence of an antigen in a sample by identifying the antigen bound to a SAM-bound antibody based on peaks observed in a mass spectrum obtained by SAMDI. In another aspect, methods of determining the concentration of an antigen in a sample are provided by adding test solutions comprising a predetermined concentration of a known antigen to the sample prior to performing SAMDI. The test solutions are added to the sample to competitively bind to one or more antibodies bound to a plurality of SAMs and analysis of the mass spectra obtained therefrom can be analyzed to determine the concentration of an antigen in the sample. In a third aspect, methods of detecting two or more antigens are provided by binding two or more different antibodies to SAMs and contacting the bound antibodies with a sample prior to analysis by SAMDI to identify the presence of the two or more antigens bound to the SAM-bound antibodies. In a fourth aspect, methods of diagnosing one or more medical conditions indicated by post-translational modification of a protein. For example, a method of diagnosing multiple sclerosis is provided that includes binding an antibody to cystatin C to a SAM, contacting the bound cysC antibody with a sample and performing SAMDI to identify the presence of a cysC antigen in the sample. In a fifth aspect, a method of diagnosing thalassemia is provided that includes binding an antibody to hemoglobin A (hem) to a SAM, contacting the bound hem antibody with a sample and performing SAMDI to identify the presence of alpha and beta subunits of the hemoglobin antigen in the sample.

In a third embodiment, biochips and methods of making biochips are provided. The methods typically include contacting a portion of a biochip surface with a SAM, contacting the SAM with a protein including a capture tag or a receptor protein and antibody, binding the protein or the receptor protein and an antibody to the SAM, and contacting the resulting SAM with a sample. In one aspect, the method includes contacting a biochip surface-bound SAM with a receptor protein and contacting the bound receptor protein with a mixture of a first antibody and a second antibody to bind both antibodies to different molecules of the SAM. The SAM may optionally include a capture ligand such as triazacyclononane (aza) to bind the receptor protein to the SAM. The receptor protein is preferably an Fc binding protein, such as Protein A or Protein G. In another aspect, biochips are provided comprising a surface-bound SAM bound to one or more antibodies, such as antibodies for cycC, hem, HSA and transferrin. The SAM preferably includes an alkanethiol moiety and an oligo(ethylene glycol) moiety.

### BRIEF DESCRIPTION OF THE FIGURES

FIG. 1A is a schematic showing the binding of a capture tag-labeled protein to a capture ligand at the distal end of an oligo(ethylene glycol) alkanethiol SAM.

FIG. 1B is a SAMDI spectrum of a 18 kDa His-tagged protein immobilized via the Ni²⁺ interaction of the capture ligand of a SAM.

FIG. 1C is a first SAMDI spectrum obtained from a second protein interacting with a first protein bound to a SAM capture ligand.

FIG. 1D is a second SAMDI spectrum obtained from a second protein interacting with a first protein bound to a SAM capture ligand.

FIG. 1E is a third SAMDI spectrum obtained from a second protein interacting with a first protein bound to a SAM capture ligand.

FIG. 1F is an interaction map for a 30×30 array of proteins performed in two orientations indicating symmetrical diagonal signatures for the interaction of certain proteins.

FIG. 2 is a schematic of a method for the formation of a SAMDI-based immunosensor. The strategy begins with a maleimide terminated monolayer that is used to immobilize the aza ligand. To immobilize the antibody, the monolayer is treated with Ni²⁺, recombinant oligohistidine Protein G (or Protein A), and an appropriate IgG antibody. The monolayer is then treated with humoral fluids (CSF, serum, plasma) containing an internal standard and analyzed with SAMDI. In the schematic, EG3 refers to tri(ethylene glycol).

FIG. 3A is a first SAMDI spectrum showing the immobilization of the Protein A onto a monolayer presenting the aza/Ni²⁺ ligand.

FIG. 3B is a second SAMDI spectrum showing the immobilization of the Protein G onto a monolayer presenting the aza/Ni²⁺ ligand.

FIG. 3C shows the immobilization of cysC antibody onto the Protein A derivatized surface.

FIG. 3D shows the immobilization of cysC antibody onto the Protein G derivatized surface.

FIG. 3E shows the capture of the cysC on a first immunosensor.

FIG. 3F shows the capture of the cysC on a second immunosensor.

FIGS. 4A-4E are SAMDI spectra for the detection of antigens from human serum using monolayers presenting antibodies for cysC (FIG. 4A), hem (FIG. 4B), HSA (FIG. 4C), and transferrin (FIG. 4D). Each spectrum reveals prominent peaks for the intended analyte and lower intensity peaks for Protein G (*) and unidentified components derived from the stock antibody (#). A control experiment using an identical monolayer that was not treated with Ni²⁺, Protein G or antibody prior to incubation with serum revealed little binding of serum components (FIG. 4E).

FIG. 5 is a SAMDI spectra for a monolayer presenting four antibodies used to simultaneously detect the four antigens cysC, hem, HSA, and transferrin from serum. The SAMDI spectrum reveals peaks corresponding to multiple charge states of the antigens and the proteins of the surface.

FIGS. 6A-6D show SAMDI spectra for the endogenous cysC in CSF quantitated through the use of a truncated form of cysC (_{trunc}cysC) as an internal standard. Several identical samples of CSF were spiked with the internal standard at concentrations ranging from 0.16-1.3 μM. These samples were applied to separate immunosensors to obtain SAMDI spectra in (FIG. 6A). Integration of the peak areas provides a quantitative measure of the ratio of the cysC to the internal standard (FIG. 6B). The competition assay at 0.49 μM (FIG. 6C) and 0.24 μM internal standard (FIG. 6D) was performed on different days, using monolayers having a range of density of the aza/Ni²⁺ ligand 0.5% (-), 1% ( - - - ), and 4%. The comparison of the peak ratios in the SAMDI spectra show the assays were highly reproducible.

FIG. 7 shows SAMDI spectra from an assessment of in vivo species of cysC in patients with multiple sclerosis. SAMDI spectra show the capture of cysC from normal human plasma, normal human serum, two control CSF samples presenting other neurological diseases, and seven CSF samples from patients with multiple sclerosis.

FIG. 8A and FIG. 8B are SAMDI spectra obtained from a SAM with an IDA capture ligand bound to a maleimide terminated SAM. FIG. 8A shows the IDA loaded maleimide alkanethiolate SAM. The antibody was bound to the IDA-transition metal cation complex and an antibody for albumin was bound to this transition metal cation. This antibody terminated SAM was free of a receptor protein. FIG. 8B shows the antibody and albumin observed using SAMDI analysis of the albumin antigen bound to the antibody.

### DETAILED DESCRIPTION

Methods and biochips useful in performing SAMDI to perform assays of enzyme activities and protein-protein interactions are provided, as well as methods of making the biochips. Significantly, these methods and devices may provide label-free detection of proteins found in clinical samples. The design and preparation of self-assembled monolayers that are designed for specific applications with SAMDI offers a straightforward method for analyzing protein variants and post-translational modifications in complex samples and holds much promise in phenotyping of proteins in diseased samples or across patient populations. As used herein, unless otherwise indicated, complex sample refers to fluids obtained from a subject, human or animal. Preferably, the humoral fluid is a clinical sample obtained from a human subject and tested by SAMDI. The extension of SAMDI from detecting low molecular weight species to large molecular weight proteins, and from analyzing solutions of defined and relatively simple composition to complex samples such as bodily fluids, cell lysates or tissue lysates, is described herein. Complex samples include humoral fluids or other clinical samples obtained, isolated or derived from a living subject (e.g., humans, plants or animals). Complex samples may be isolated from a living subject using any suitable technique, including lysis of cells obtained from the subject. Examples of complex samples include single cellular organisms (e.g., bacteria), cell lysates, tissue lysates, tumor lysates, and subcellular organelles (e.g., mitochondria, ribosomes and the like).

Certain preferred methods below describe SAMDI analysis of large molecular weight protein analytes (e.g., 25 kDa-200 kDa) and provide the examples of monolayer (SAM) surfaces able to discriminate an antigen from the many other components in complex samples in diagnostic assays. By using SAMDI-based techniques and methods, the interaction of the first protein and the second protein is analyzed in a manner that does not require detection of a fluorescent reporter group, a radioactive reporter group or a heterologous tag that is adapted to bind a fluorescent or enzymatic reporter. These and other related embodiments are described in detail below. The self-assembled monolayers for matrix assisted laser desorption ionization mass spectrometry (SAMDI) may employ any suitable mass spectrometry means for detecting desorbed (i.e., ablated) species. Although time of flight (TOF) is the mass analyzer is preferably used to obtain the mass spectra during the SAMDI technique, SAMDI may be performed using a variety of mass analyzers (quadrupole, ion-trap, Fourier transform mass spectroscopy (FTMS), TOF or hybrid combinations of these) to obtain a mass spectra having mass to charge peaks useful in observing ablated ionized species obtained from the SAM.

### Protein-Protein Interaction

In a first embodiment, methods for observing, identifying, characterizing and/or mapping protein-protein interactions are provided. This work is based on combining surface chemistries and immobilization chemistries to provide for high activity of immobilized proteins and low levels of non-specific background adsorption (which remains a technical difficulty associated with current microarray work).

The methods described herein provide protocol for mapping protein interactions that does not require the use of labeled molecules for detection. Using the preferred analytical methods and SAMs disclosed herein, protein-protein interactions may be analyzed in a manner that does not require detecting a fluorescent reporter group, a radioactive reporter group or a heterologous tag that is adapted to bind a fluorescent or enzymatic reporter. However, the methods may optionally be practiced in combination with known reporter molecule detection methods. Using the methods described herein, mass spectrometry methods can directly observe the proteins and are therefore especially valuable in these applications. The biomolecular interactions observed using SAMDI methods and biochips described herein may include not just single interactions between two biomolecules, but also interactions within a protein complex (e.g., interaction between the alpha and beta subunits of Hemoglobin A), as well as interactions between more than two biomolecules (e.g., interaction between proteinG receptor protein bound to a SAM, an antibody bound to the receptor protein and an antigen bound to the bound antibody).

The protein-protein interactions may be measured within a set of recombinantly expressed proteins. The approach is based on the use of self-assembled monolayers (SAMs) that can selectively immobilize appropriately tagged proteins, and the use of matrix-assisted laser desorption/ionization mass spectrometry to observe the surface-bound proteins. Interaction between a first protein and a second protein can be measured using a self-assembled monolayer adapted to selectively bind the first protein comprising a capture tag in the presence of the second protein without the capture tag. The term “capture tag” refers generally to any portion of a molecule capable of binding to a capture ligand, rather than to a particular structure of the capture tag. For example, a protein comprising a capture tag may be capable of binding to a capture ligand of a SAM using any suitable structure. While the protein comprising the capture tag may optionally include a recombinant tag associated with the protein that binds to the capture ligand, the recitation of a protein with a “capture tag” does not require a recombinant tag and many embodiments are performed using capture tag containing proteins that do not include such a recombinant tag. Preferably, the capture tag is selected to provide specific binding of the capture tag to a capture ligand on a SAM with a binding constant high enough to retain the molecule comprising the capture ligand bound to the SAM for an intended application, such as SAMDI detection of the bound molecule comprising the capture tag. For instance, the capture tag may be selected to covalently bind to the capture ligand of a surface bound SAM. Similarly, the term “tag” refers to a portion of a molecule that is capable of binding or detection.

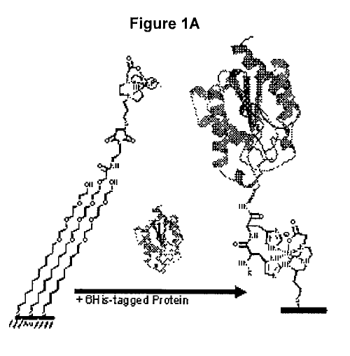

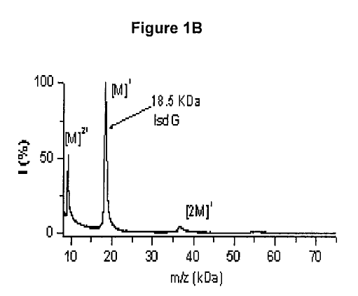

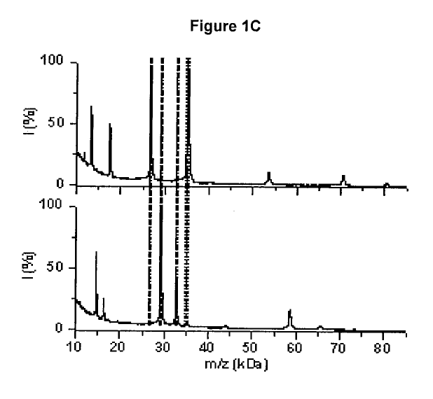

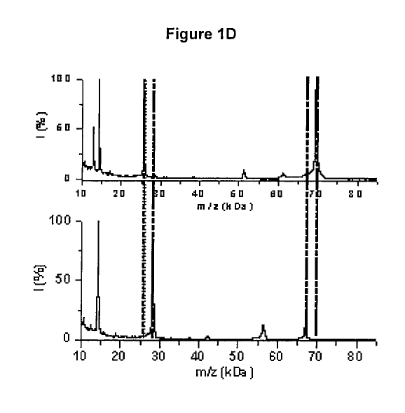

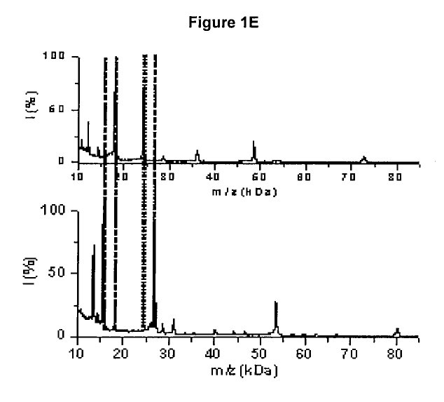

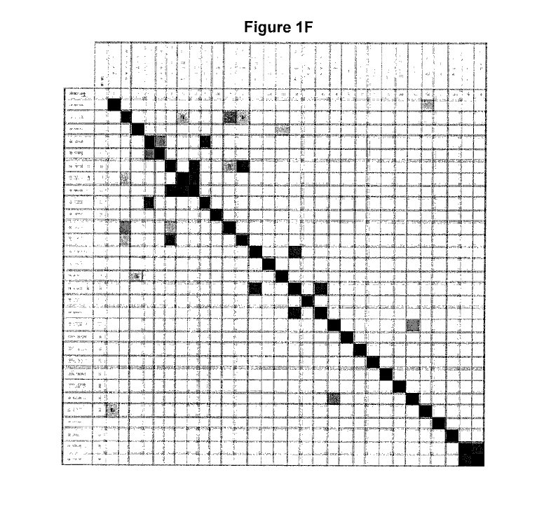

Methods for determining protein binding partners preferably use a suitable capture ligand immobilized to a SAM. FIG. 1A is a schematic showing the binding of a first protein (a capture tag-labelled protein) to a capture ligand at the distal end of an oligo(ethylene glycol) alkanethiol SAM. The loading of the capture ligand on the SAM may be varied. Preferably, the loading is selected as a percentage of the SAM molecules for an intended application. Importantly, the loading of the capture ligand may be lower in SAMDI techniques using a SAM that reduces non-specific binding to the surface than for other analytical techniques. SAM's presenting capture ligands that selectively bind a protein such as an antibody permit a high percent of the bound molecules to function during the SAMDI analysis. Accordingly, SAMDI analysis may be performed with capture ligand loadings of less than 100% of the SAM surface area, and preferably about 50% or less, and most preferably about 0.5-50%, 0.5-25%, 0.5-15% and 0.5-5%. Also significantly, the SAMDI methods exemplified herein permit the identification and observation of biomolecular binding interactions between a first protein and a second protein each having a molecular weight greater than about 25 kDa using the capture ligand loadings recited above. For example, a first protein may be an antibody with a molecular weight of about 150 kDa and the second protein may be an antigen having a molecular weight of about 80 kDa. Significantly, the methods permit detection of these interactions between biomolecules larger than about 20 kDa or about 50 kDa using SAMDI with a SAM surface having a capture ligand loading of less than 100%, including the capture ligands recited above.

In a first aspect, the first protein is mixed with one or more untagged proteins, such as the second protein, in a protein solution prior to contacting the first protein with the SAM. Desirably, the first protein and the second protein are both contacted with the SAM by contacting the protein solution with the SAM. The first protein comprising the tag may bind to the SAM. When the second protein interacts with the first protein, the second protein may bind to the fraction of the first protein bound to the SAM. The protein solution may include a plurality of untagged proteins in addition to the second protein, permitting the method to screen a wide variety of proteins simultaneously for interactive binding with the first (tagged) protein. The protein solution may be contacted with the SAM for various amounts of time to analyze the binding of the first and second proteins to each other and/or the SAM. Accordingly, the first protein and the second protein may be combined in a protein solution that is contacted with the SAM in a manner effective to permit at least a fraction of the first protein to bind to the SAM and at least a fraction of the second protein to bind to a fraction of the first protein bound to the SAM. The first protein and the second protein may be mixed for a suitable first period of time. Preferably, either the first protein or the second protein includes a capture tag adapted to bind to a capture ligand on the SAM.

Alternatively, the first protein may be contacted with the SAM in a manner effective to bind at least a fraction of the first protein comprising the capture tag to the SAM followed by contacting the second protein free of the capture tag with the SAM in a manner effective to bind the second protein to a fraction of the first protein bound to the SAM. The first protein preferably contains a capture tag and is contacted with the SAM for a first period of time sufficient to bind the first protein to the SAM by selective binding of the capture tag to a portion of the SAM. The second protein free of the capture tag may be contacted with the first protein bound to the SAM for a second period of time sufficient to bind a desired fraction of the second protein to the first protein. The first period of time may be the same or different from the second period of time. Accordingly, the first protein is contacted with the SAM in a manner effective to bind at least a fraction of the first protein to the SAM. Next, the second protein may be contacted with the first protein bound to the SAM and binding of the second protein to the bound first protein may be subsequently characterized. In addition, the second protein may be contacted with the first protein bound to the SAM as part of a protein solution containing a plurality of untagged proteins. In this aspect, the protein solution may optionally be free of the tagged first protein. The method may further comprise the steps of removing (e.g., washing) the fraction of the first protein that is not bound to the SAM, and/or removing the fraction of the second protein (or other untagged proteins) that are not bound to the fraction of the first protein bound to the SAM. The first protein and the second protein may be contacted with the SAM for various amounts of time to analyze the binding of the first and second proteins to each other and/or the SAM.

For example, a first recombinant protein comprising an N-terminal or C-terminal oligohistidine may be immobilized to the SAM and then used to capture a second protein binding partner. Possible binding partners may be tested for binding individually or in groups of two or more simultaneously. Alternatively, the first recombinant protein comprising an N-terminal or C-terminal oligohistidine tag may be mixed with one or more putative binding partners prior to immobilization to the monolayer. This may be necessary as binding of the first protein to the monolayer may interfere with capture of binding partners. Optionally, the SAM may be washed to remove protein(s) not bound to the SAM before and/or after contacting the SAM with a protein. For example, a first protein including the capture tag and a second protein without the capture tag may be removed from the SAM prior to analyzing the SAM by matrix assisted laser desorption ionization mass spectrometry (SAMDI).

The method may further comprise the step of contacting a second protein including the capture tag and the first protein free of the capture tag with a second SAM in a manner effective to bind the capture tag to the second SAM, followed by analysis of the SAM by SAMDI techniques, including analysis of a resulting mass spectrum to identify one or more mass-to-charge peaks characteristic of the second protein bound to the second SAM.

Typically, the SAMDI analysis is performed at least twice: first after contacting a second protein without a capture tag with a first SAM comprising the first protein including a capture tag bound to the SAM, followed by performing the SAMDI analysis on a second SAM that is formed by contacting the first protein without the capture tag with a second protein bound to a second SAM via a capture tag.

The SAM is preferably an alkanethiol modified to prevent non-specific binding to the surface. For example, in one aspect, the SAM includes a surface-bound alkanethiol attached to an oligo(ethylene glycol) moiety. “Non-specific adsorption” refers to the adsorption of a protein onto a surface by an interaction other than a ligand/receptor interaction. When the alkanethiol contains oligo(ethylene glycol) groups, the oligo(ethylene glycol) oligomer preferably contains 3 to 7 units. When the alkanethiol contains oligo(propylene sulfoxide) group, the oligo(propylene sulfoxide) oligomer preferably contains 3 units.

The inertness of the SAMs maximizes the activity of the immobilized ligand and reduces false signals due to non-specific interactions. Preferably, the SAM is adapted to selectively bind a capture tag, such as oligohistidine sequence, bound to a protein of interest. For example, the SAM may include a capture ligand adapted to bind to the capture tag, such as a triazacyclononane (aza) or nitrilotriacetic acid (NTA) moiety. The capture tag may be attached to any suitable portion of the SAM, such as the portion of the oligo(ethylene glycol) moiety distal to the surface bound portion of the SAM.

The capture tag typically is attached to a protein. Typically, the methods include contacting a first protein comprising the capture tag with a SAM presenting a capture ligand. For example, a capture ligand may be adapted to bind oligohistidine tagged proteins on the SAM monolayer presenting the aza/Ni²⁺ capture ligand, or the equivalent of a NTA-Ni²⁺ ligand, against a background of tri(ethylene glycol) groups arrayed a SAM.

Other immobilization chemistries may be harnessed, including the embodiments described in the Examples. In one aspect, the proteins bound to the SAM are his-tagged proteins bound to SAMs having the appropriate chemistries for immobilization of these proteins. Any other suitable immobilization tags, including cutinase, GST, oligionucleotides, antigens, carbohydrates, or any of many others, may be used provided that a surface with a corresponding partner that selectively recognizes the tag is available. These alternate methods may accommodate existing sets of recombinant proteins or be preferable in certain high throughput protein expression systems. Optionally, the first or the second protein comprises a predicted protein-protein interaction domain. Protein-protein interaction domains include, but are not limited to domains involved in phosphotyrosine binding (e.g. SH2, PTB), phosphor-serine binding (e.g. UIM, GAT, CUE, BTB/POZ, VHS, UBA, RING, HECT), Proline-rich region binding (e.g. EVH1, WW, SH3, GYF), apoptosis (e.g. BIR, TRAF, DED, Death, CARD, BH), cytoskeleton modulation (e.g. ADF, GEL, DH, CH, FH2), or other cellular functions (e.g. EH, CC, VHL, TUDOR, PUF Repeat, PAS, MH1, LRR, IQ, HEAT, GRIP, TUBBY, SNARE, TPR, TIR, START, SOCS Box, SAM, RGS, PDZ, PB1, LIM, F-BOX, ENTH, EF-Hand, SHADOW CHROMO, CHROMO, BROMO, ARM, ANK).

The interaction of the first protein and the second protein may be analyzed by SAMDI techniques. Matrix-assisted laser desorption/ionization and time of flight mass spectrometry (e.g., SAMDI) can be used to characterize SAMs. The SAMDI technique is based on the discovery in the late 1980s that desorption/ionization of large, nonvolatile molecules such as proteins can be effected when a sample of such molecules is irradiated after being codeposited with a large molar excess of an energy-absorbing “matrix” material, even though the molecule does not strongly absorb at the wavelength of the laser radiation. The abrupt energy absorption initiates a phase change in a microvolume of the absorbing sample from a solid to a gas while also inducing ionization of the sample molecules. Detailed descriptions of the MALDI-TOF-MS technique and its applications may be found in review articles by E. J. Zaluzec et al. (Protein Expression and Purification, Vol. 6, pp. 109-123 (1995)) and D. J. Harvey (Journal of Chromatography A, Vol. 720, pp. 429-4446 (1996)), each of which is incorporated herein by reference. In brief, the matrix and analyte may be mixed to produce a solution with a matrix:analyte molar ratio of approximately 10,000:1. A small volume of this solution, typically 0.5-2 μl, is applied to a stainless steel probe tip and allowed to dry. During the drying process the matrix codeposits from solution with the analyte. The analyte may include a protein, protein complex, antibody, antigen or other biomolecule or modified biomolecule being detected in a sample.

Ionization of the analyte may be effected by pulsed laser radiation focused onto the probe tip which is located in a short (about 5 cm) source region containing an electric field. The ions formed at the probe tip are accelerated by the electric field toward a detector through a flight tube, which is a field free drift region. Since all ions receive the same amount of energy, the time required for ions to travel the length of the flight tube is dependent on their mass. Thus, low-mass ions have a shorter time of flight (TOF) than heavier ions. All the ions that reach the detector as the result of a single laser pulse produce a transient TOF signal. Typically, ten to several hundred transient TOF mass spectra are averaged to improve ion counting statistics.

The mass of an unknown analyte is determined by comparing its experimentally determined TOF to TOF signals obtained with ions of known mass. The SAMDI technique is capable of determining the mass of proteins of between 1 and 40 kDa with a typical accuracy of +/− 0.1%, and a somewhat lower accuracy for proteins of molecular mass above 40 kDa.

Optionally, a matrix may be applied to the SAM with one or more bound protein(s) prior to the ionization and ablation step. Examples of suitable matrix materials include benzoic acids, and derivatives thereof. One preferred matrix is 2,5-dihydroxyl benzoic acid. The matrix can be applied by delivering a solution containing the matrix to the metal surface. The concentration of the matrix can vary; typically it is between 1 and 50 mg/mL. The solvent can vary; typically it is acetonitrile, water, acids, or an alcohol (such as ethanol, methanol, isopropanol, etc.). Other matrix compositions include sinapinic acid (SA) or α-cyano-4-hydroxycinnamic acid (CHCA) at 5-7.5 mg/ml (in acetone, 0.3 uL manually spotted), which may be used to facilitate protein desorption and ionization from the monolayer surface.

Advances in mass spectrometry equipment can lead to improved sensitivities—which can significantly reduce the time required to acquire mass spectra, and therefore increase the number of interactions that can be read per unit time. These higher performance instruments also provide superior control over the laser sources that are used to desorb proteins from the surface, and therefore can be used to read higher density arrays. Typically, the SAM comprising the bound first and/or second protein(s) may be laser ionized and ablated to obtain a mass spectrum including at least one mass-to-charge peak characteristic of the first protein bound to the SAM. SAMDI analysis of these monolayers allows for the determination of whether the first protein interacts with any of the possible binding partners. In order to confirm the protein-protein interactions identified by this method, the reciprocal assay is performed where the possible binding partner is immobilized to the array and the capture of the first protein, lacking a oligohistidine tag, is assayed by SAMDI. Optionally, one of the first protein or the second protein may be an aqueous-soluble portion of an aqueous-insoluble peptide expressed by an open reading frame of a gene.

One preferred method of characterizing the interaction of a first protein with a second protein using a self assembled monolayer (SAM) on a biochip and matrix assisted laser desorption/ionization and time of flight mass spectrometry (self-assembled monolayers for matrix assisted laser desorption ionization mass spectrometry or SAMDI) comprises the steps of: mixing a first protein including a capture tag and a second protein free of the capture tag to form a first protein solution; contacting the first protein solution with a first SAM in a manner effective to bind the capture tag to the first SAM, the first SAM comprising a capture ligand that selectively binds to the capture tag; and analyzing the first SAM comprising the bound first protein and any of the second protein bound to the first protein bound to the SAM by SAMDI. The first protein preferably has a mass of about 5,000 to 200,000 Daltons and the SAM is preferably a capture-ligand-terminated oligo(ethylene glycol) alkanethiol. Optionally, the method further includes the steps of removing the first protein including the capture tag that is not bound to the first SAM and the second protein that is not bound to the fraction of the first protein bound to the first SAM prior to analyzing the first SAM by SAMDI after removing the fraction of the first protein that is not bound to the first SAM and the second protein that is not bound to the fraction of the first protein bound to the first SAM.

The method may further include one or more of the following steps: mixing the second protein including a capture tag and the first protein free of the capture tag to form a second protein solution; contacting the second protein solution with a second SAM in a manner effective to bind the capture tag to the second SAM, the second SAM comprising a capture ligand that selectively binds to the capture tag; analyzing the second SAM comprising the bound second protein and any of the first protein bound to the second protein bound to the SAM by SAMDI; removing the first protein including the capture tag that is not bound to the first SAM and the second protein that is not bound to the first protein bound to the first SAM prior to performing SAMDI on the first SAM; and removing the second protein including the capture tag that is not bound to the second SAM and the first protein that is not bound to the second protein bound to the second SAM prior to performing SAMDI on the second SAM.

Certain non-limiting examples of preferred embodiments relating to measurement of protein-protein interactions are enumerated below.

In one exemplary aspect, a method of determining or observing biomolecular interactions between a first protein and a second protein using a self-assembled monolayer (SAM) adapted to selectively bind the first protein comprising a capture tag in the presence of the second protein without the capture tag, the method comprising the steps of: (a) contacting a first protein including the capture tag and a second protein free of the capture tag with the SAM in a manner effective to bind the capture tag to the SAM; (b) ionizing and ablating the SAM, the first protein bound to the SAM and any of the second protein bound to the first protein bound to the SAM and obtaining a mass spectrum including at least one mass-to-charge peak characteristic of the first protein bound to the SAM; and (c) analyzing the mass spectrum to determine if the second protein interacted with the first protein bound to the SAM. Step b may include applying a matrix to the SAM prior to ionizing and ablating the SAM. The matrix may include a benzoic acid, or a derivative thereof. Preferably, step b. is performed by matrix assisted laser desorption ionization mass spectrometry.

The first protein may be contacted with the SAM in a manner effective to bind at least a fraction of the first protein comprising the capture tag to the SAM followed by contacting the second protein free of the capture tag with the SAM in a manner effective to bind the second protein to a fraction of the first protein bound to the SAM. Alternatively, the first protein and the second protein may be mixed to form a protein solution, followed by contacting the protein solution with the SAM in a manner effective to permit at least a fraction of the first protein to bind to the SAM and at least a fraction of the second protein to bind to a fraction of the first protein bound to the SAM.

Optionally, the method may further comprise additional steps. For example, the method may further comprise the step of removing the first protein including the capture tag that is not bound to the SAM and the second protein that is not bound to the first protein bound to the SAM before performing step b. The method may also include the step(s) of contacting the second protein including the capture tag and the first protein free of the capture tag with a second SAM in a manner effective to bind the capture tag to the second SAM; and/or ionizing and ablating the second SAM, the second protein bound to the second SAM and any of the first protein bound to the second protein bound to the second SAM and obtaining a second mass spectrum with at least one mass-to-charge peak characteristic of the second protein bound to the second SAM; and/or analyzing the second mass spectrum to characterize the interaction of the first protein with the second protein bound to the second SAM. In addition, the method may further comprise the step of removing the second protein including the capture tag that is not bound to the second SAM and the first protein that is not bound to the second protein bound to the second SAM.

Preferably, the method also includes observing and/or characterizing the interaction of the first protein and the second protein by comparing the first mass spectrum and the second mass spectrum to characterize the binding of: (a) the second protein free of the capture tag to the first protein with the capture tag; and/or (b) the first protein free of the capture tag to the second protein with the capture tag.

The combined structure of an immobilized protein bound to the surface of a SAM and portions thereof may have various structures. For example, the protein may include an oligohistidine sequence tag, such as a hexahistidine, or a histidine rich region. The SAM may include an oligo(ethylene glycol) alkanethiol adapted to selectively bind the capture tag. For example, the SAM may include a capture ligand such as triazacyclononane (aza), an aminodiacetic acid (IDA) or a nitrilotriacetic acid (NTA).

In another exemplary embodiment, methods are provided for characterizing the interaction of a first protein with a second protein using a self-assembled monolayer (SAM) on a biochip and matrix-assisted laser desorption/ionization and time of flight mass spectrometry (SAMDI) comprising the steps of: (a) mixing a first protein including a capture tag and a second protein free of the capture tag to form a first protein solution; (b) contacting the first protein solution with a first SAM in a manner effective to bind the capture tag to the first SAM, the first SAM comprising a capture ligand that selectively binds to the capture tag; and (c) analyzing the first SAM comprising the bound first protein and any of the second protein bound to the first protein bound to the SAM by SAMDI.

Optionally, these methods may further comprise one or more of the following steps: d. mixing the second protein including a capture tag and the first protein free of the capture tag to form a second protein solution; e. contacting the second protein solution with a second SAM in a manner effective to bind the capture tag to the second SAM, the second SAM comprising a capture ligand that selectively binds to the capture tag; f. analyzing the second SAM comprising the bound second protein and any of the first protein bound to the second protein bound to the SAM by SAMDI. Alternatively, these methods may further comprise one or more of the following steps: d. removing the first protein including the capture tag that is not bound to the first SAM and the second protein that is not bound to the first protein bound to the first SAM prior to performing SAMDI on the first SAM; and/or f. removing the second protein including the capture tag that is not bound to the second SAM and the first protein that is not bound to the second protein bound to the second SAM prior to performing SAMDI on the second SAM. At least one of the first SAM and the second SAM may include an oligo(ethylene glycol) alkanethiol capture ligand adapted to selectively bind the capture tag.

Preferably, the interaction of the first protein and the second protein is analyzed in a manner that does not require detecting a fluorescent reporter group, a radioactive reporter group or a heterologous tag that is adapted to bind a fluorescent reporter. Methods are provided for characterizing the interaction of a first protein having a mass of about 5,000 to 200,000 Daltons with a second protein using a capture-ligand-terminated oligo(ethylene glycol) alkanethiol self-assembled monolayer (SAM) on a biochip and matrix-assisted laser desorption/ionization and time of flight mass spectrometry (SAMDI) comprising the steps of: (a) contacting a first protein tagged with an N-terminal oligohistidine sequence capture tag and a second protein free of the N-terminal oligohistidine sequence capture tag with a first SAM in a manner effective to bind the capture tag of at least a fraction of the first protein to the first SAM; (b) removing the first protein including the capture tag that is not bound to the first SAM and the second protein that is not bound to the fraction of the first protein bound to the first SAM; (c) analyzing the first SAM by SAMDI after removing the fraction of the first protein that is not bound to the first SAM and the second protein that is not bound to the fraction of the first protein bound to the first SAM; (d) contacting the second protein tagged with an N-terminal oligohistidine sequence capture tag and the first protein free of the N-terminal oligohistidine sequence capture tag with a second SAM in a manner effective to bind the capture tag of at least a fraction of the second protein to the second SAM; (e) removing the second protein including the capture tag that is not bound to the second SAM and the first protein that is not bound to the fraction of the second protein bound to the second SAM; and (f) analyzing the second SAM by SAMDI after removing the fraction of the second protein that is not bound to the second SAM and first protein that is not bound to the fraction of the second protein bound to the second SAM. Step (a) may include mixing the first protein including the capture tag and the second protein free of the capture tag together to form a first protein solution followed by contacting the first protein solution with the first SAM. Step (d) may include mixing the second protein including the capture tag and the first protein free of the capture tag together to form a second protein solution followed by contacting the second protein solution with the second SAM. In some examples, the molecular weight of protein 1 and/or protein 2 may be selected within a mass range of about 25,000-200,000 Daltons, 25,000-175,000 Daltons, 25,000-150,000 Daltons, 25,000-125,000 Daltons, 25,000-100,000 Daltons, 50,000-200,000 Daltons, 50,000-175,000 Daltons, 50,000-150,000 Daltons, 50,000-125,000 Daltons, and 50,000-100,000 Daltons. The SAM may have less than 100% loading of the capture ligand, protein and/or antibody, including loadings of about 0.5-50%, 0.5-25%, 0.5-15%, 0.5-10% and 0.5-5%. Importantly, the interaction of the first protein and the second protein is preferably analyzed in a manner that does not require detecting a fluorescent reporter group, a radioactive reporter group or a heterologous tag that is adapted to bind a fluorescent reporter.

In yet another exemplary embodiment, methods of characterizing interaction between a first protein and a plurality of analyte proteins is provided using a self-assembled monolayer (SAM) adapted to selectively bind the first protein comprising a capture tag in the presence of one or more of the plurality of analyte proteins that are free of the capture tag, the method comprising the steps of: (a) independently contacting a first protein including the capture tag with two or more analyte proteins selected from a plurality of analyte proteins, the analyte proteins being free of the capture tag, the first protein and the analyte proteins each contacted with a SAM in a manner effective to bind the capture tag to the SAM; (b) ionizing and ablating the SAM, the first protein bound to the SAM and any of the analyte proteins bound to the first protein bound to the SAM and obtaining a mass spectrum including at least one mass-to-charge peak characteristic of the first protein bound to the SAM; and (c) analyzing the mass spectrum to determine if the second protein interacted with the first protein bound to the SAM. Preferably, the interaction of the first protein and the second protein is analyzed in a manner that does not require detecting a fluorescent reporter group, a radioactive reporter group or a heterologous tag that is adapted to bind a fluorescent reporter. In one aspect, at least one of the first protein and the second protein is an aqueous-soluble protein. For example, at least one of the first protein and the second protein may be a portion of a peptide expressed by an open reading frame of a gene, or at least one of the first protein and the second protein comprises a predicted protein-protein interaction domain.

### Immunoassays

In a second embodiment, methods for performing immunoassays using antibodies attached to SAMs in combination with SAMDI analysis are provided. Preferred immunoassay methods of detecting an antigen in a sample include the use of a first antibody bound to a self-assembled monolayer (SAM). The preferred immunoassay methods permit use of SAMDI to analyze large molecular weight protein analytes in complex samples from living subjects, and provide diagnostic assays capable of discriminating an antigen from other components in complex samples.

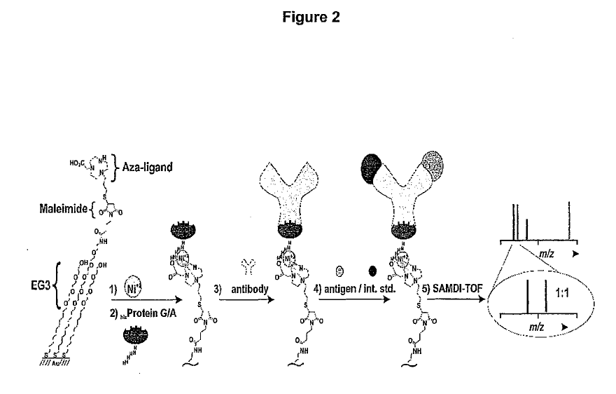

FIG. 2 is a schematic showing the use of a preferred SAM structure and method for immobilizing an antibody bound to a SAM, and subsequently using SAMDI to detect the presence of an antigen bound to the surface-bound SAM-antibody structure. The SAM is preferably an alkanethiol modified to prevent non-specific binding to the surface. The SAM preferably includes a surface-bound alkanethiol attached to an oligo(ethylene glycol) moiety. The method may be based on immobilization of oligohistidine tagged proteins and may include a SAM that presents the aza/Ni²⁺ ligand at a density of 0.5-5% (relative to total alkanethiolate) against a background of tri(ethylene glycol) groups. The aza/Ni²⁺ complex is an alternative to the commonly used NTA ligands and has been described for immobilization of proteins to SAMs.

Preferably, the SAM is adapted to selectively bind an antibody. For example, the SAM may include a receptor protein, such as an Fc binding protein (e.g., Protein A or Protein G). The SAM may include a capture ligand adapted tobind to the receptor protein, such as a triazacyclononane (aza), nitrilotriacetic acid (NTA), or iminodiacetic acid (IDA) coordinated with transition metals.

In some embodiments, a SAM may include a receptor protein such as Protein G (or Protein A) to immobilize an antibody to the SAM monolayer. This strategy can be applied to a broad range of immunoassays, provided that an IgG type antibody with reasonable affinity and selectivity for the intended analyte is available. The specific interaction between Protein G/A and the Fc region of the antibody provides a uniform orientation of the antibody and control over the density of the antibody (which is determined by the density of the Aza ligand used to immobilize the Protein G/A). Alternatively, cutinase-mediated immobilization may be used to give oriented immobilization of antibodies (Kwon, Y.; Han, Z. Z.; Karatan, E.; Mrksich, M.; Kay, B. K. Analytical Chemistry 2004, 76, 5713-5720). Third, the assay format reported here does not require modification of either the antibody or the analyte, although a maleimide functionalized SAM supports direct attachment of Fab or Fab' fragments generated after enzymatic digestion of antibodies. Other examples that do not include use of a receptor protein to immobilize the antibody include: streptavidin to immobilize biotinylated antibodies, or the iminodiacetic acid (IDA) ligand to capture the naturally occurring histidine rich region commonly found in antibodies.

The antibody may be selected to bind to an antigen that is indicative of a medical condition. For example, an antigen may be compared to a truncated or chemically modified version of the antigen having a different mass to charge peak in a MS analysis using SAMDI. Quantitative comparison of post-translationally derived biomarkers or protein variants using SAMDI may yield valuable information about the stability or expression of the variants or post-translational modifications (PTMs) and prove to be diagnostically useful, as has been highlighted by previous groups when studying proteins present in blood-(Nedelkov, D.; Kiernan, U. A.; Niederkofler, E. E.; Tubbs, K. A.; Nelson, R. W. Proceedings of the National Academy of Sciences of the United States of America 2005, 102, 10852-10857; Shimizu, A.; Nakanishi, T.; Miyazaki, A. Mass Spectrometry Reviews 2006, 25, 686-712; Griffith, W. P.; Kaltashov, I. A. CurrentmOrganic Chemistry 2006, 10, 535-553). By comparing the ratio of two forms of the same antigen, the need for an internal standard is reduced.

The SAM-bound antibodies may be used in SAMDI methods of diagnosing one or more medical conditions indicated by post-translational modification of a protein. In addition, genetic mutations such as single nucleotide polymorphisms (SNPs) and gene splicing on DNA can result in new amino acids or deletions of amino acids that can result a mass shift detectable by SAMDI. In addition, certain hemoglobinopathies such as sickle cell may be detectable by a change in mass during SAMDI analysis.

Post translational modifications that may be detected by SAMDI include, without limitation, the following modifications to an antigen or SAM-bound protein: Acetylation, ADP-ribosylation, Amidation, Beta-methylthiolation, Biotinylation, Bromination, Carboxylation, Citrullination, C-Mannosylation, Cysteinylation, Deamination, Diacylglycerol modification, Dimethylation, FAD modification, Farnesylation, Hydroxyfarnesylation, FMN conjugation, Formylation, Geranyl-geranylation, Glycosylation, Glutathionylation, Hydroxylation, Lipidation, Lipoic acid modification, Methylation, Myristolation, S-nitrosylation, palmitoylation, phosphorylation, Pyruvic acid modification, Stearoylation, Sulfation, SUMOylation, Trimethylation, Ubiquitination and proteolytic cleavage. Accordingly, SAMDI may be used to detect post-translationally modified proteins as antigens that are indicative of medical conditions by binding these antigens to a SAM-bound antibody and detecting the bound modified proteins by SAMDI. Examples of medical conditions believed to be indicated by the presence of post-translationally modified proteins include, without limitation:
1) Angleman syndrome, Liddle's syndrome, and von Hippel-Landau syndrome (Ubiquitination)
2) Asthma, chronic obstructive pulmonary disease, many cancer types (histone post-translational modifications)
3) Huntington's disease, polyglutamine diseases (acetylation)
4) Alzheimer's disease (alpha-amidation of neuropeptides)
5) Coagulation factor abnormalities (gamma-carboxylation)
6) Multiple Sclerosis (histone citrullination)
7) Rheumatoid arthritis, Alzheimer's, psoriasis, autoimmune diseases, multiple sclerosis, psoriasis, glaucoma, adenocarcinomas (citrullination)
8) Leukocyte adhesion deficiency II, Alzheimer's disease, paroxysmal nocturnal hemoglobinuria, lysomal diseases (aspartylglycosaminuria, alpha- & beta-mannosidosis, gangliosidosis, sialidosis, fucosidosis), CDG-Syndromes (currently 12 disease groups), cystic fibrosis, I-Cell disease, congential dyserythropoietic anemia type II, Wiskott-Aldrich syndrome, glycoproteinoses, diabetic cardimyopathy (glycosylation)
9) Parkinson's disease (dopamine deamination)
10) Spinal muscular atrophy (dimethylation)
11) Batten disease, progeroid syndromes (farnesylation)
12) Choroideremia, Hermansky-Pudlak syndrome, Griscelli syndrome (Geranylgeranylation)
13) Friedreich's Ataxia, hyperlipidemia, chronic renal failure, type I & II diabetes, mycosis fungoides (Glutathionylation)
14) Cardiovascular disease (lipidation)
15) Brittle bone disease, Parkinson's disease (hydroxylation)
16) Sporadic Parkinson's disease, pulmonary hypertension, diabetes, ischemia, asthma, hypercholesterolemia (S-nitrosylation)
17) Osteoarthritis, microalbuminuria, cystic fibrosis, cancer, chondrodysplasia (sulfation); and
18) Neuronal intranuclear inclusion disease, polyglutamine diseases, Huntington's disease, Alzheimer's disease, type 1 diabetes (SUMOylation).
19) Multiple sclerosis (cysC proteolytic cleavage)

The preferred immunoassay methods may be performed in the absence of SPR or labeling of the antibody or antigen because SAMDI can observe the antigen directly. Many immunosensor schemes, for example, require chemical or enzymatic modification of the Fc region of the antibody to introduce functionality needed for the immobilization, and additionally require labeling of either antibody or antigen to detect the latter. The preferred immunoassays do not require two antibodies with high affinity and non-overlapping epitopes on the antigen, as the use of SAMDI abrogates the need for the second antibody and substantially streamlines the assay development cycle.

The capture ligand may be attached to any suitable portion of the SAM, such as the portion of the oligo(ethylene glycol) moiety distal to the surface bound portion of the SAM. Typically, the methods include contacting a first antibody with a SAM presenting a receptor protein bound to the SAM. For example, a SAM presenting an Fc binding protein may be contacted with a solution comprising one or more antibodies in a manner effective to bind at least a first antibody to the binding protein. Recombinant Protein G (or Protein A) containing a oligohistidine tag is immobilized to the monolayer and then used to capture an IgG antibody by way of binding to the heavy chain constant region of the antibody (Fc). The specific interactions that mediate the immobilization enforce a defined orientation of the antibody on the surface while not interfering with the antigen binding region on the antibody and therefore are expected to optimize the activity of the immobilized antibody. Further, this strategy offers control over the density of antibody, which can be particularly relevant to reducing the steric interactions of large antigens at the surface. The bound first antibody and SAM may be contacted with a sample in a manner effective to bind an antigen in the sample to the bound first antibody.

Optionally, the SAM may also be contacted with two or more antibodies in a manner effective to provide competitive binding between the first and second antibodies with the receptor protein(s) on the SAM.

The SAM comprising the bound first antibody and any antigen bound to the bound first antibody may be ionized and ablated to obtain a mass spectrum by SAMDI techniques, as described above, to obtain a mass spectrum. The mass spectrum may include at least one mass-to-charge peak characteristic of the bound first antibody, one or more peaks characteristic of the antigen bound to the bound first antibody.

Certain non-limiting examples of preferred immunoassay embodiments are enumerated below.

In one exemplary embodiment, an immunoassay method of detecting an antigen in a sample is provided using a first antibody bound to a self-assembled monolayer (SAM), the method comprising the steps of: (a) contacting a SAM including a receptor protein with the first antibody in a manner effective to bind the first antibody to the receptor protein to form a bound first antibody; (b) contacting the bound first antibody with the sample in a manner effective to bind a first antigen in the sample to the first antibody; (c) ionizing and ablating the SAM, the bound first antibody and any of the first antigen bound to the bound first antibody and obtaining a mass spectrum including at least one mass-to-charge peak characteristic of the bound first antibody; and (d) analyzing the mass spectrum to determine if the first antigen attached to the bound first antibody. Preferably, the binding of the antigen to the bound first antibody is analyzed in a manner that does not require detecting a fluorescent reporter group, a radioactive reporter group or a heterologous tag that is adapted to bind a fluorescent reporter.

Optionally, step (a) includes contacting the SAM with a solution comprising the first antibody. Step (b) may include contacting the SAM including the bound first antibody with a liquid sample containing the first antigen, and may further include removing the sample, including a first antigen that is not bound to the bound first antibody, from the SAM after contacting the bound first antibody with the first antigen in the sample. Step (c) may be performed by matrix assisted laser desorption ionization mass spectrometry, and may also include applying a matrix to the SAM prior to ionizing and ablating the SAM. Optionally, the method further comprises the step of binding the receptor protein to the SAM prior to contacting the SAM with the first antibody, the step performed by contacting a SAM comprising a capture ligand with the receptor protein in a manner effective to bind the capture ligand to the receptor protein. The SAM may include a receptor protein. When present, the receptor protein comprises an Fc binding protein. For example, the Fc binding protein may include Protein A, Protein G or a combination thereof. Optionally, the SAM is attached to a biochip. The method may further include the step of contacting the SAM with a second antibody in a manner effective to bind the second antibody to the receptor protein to form a bound second antibody, the step performed prior to ionizing and ablating the SAM. Step (a) may include contacting the SAM with a solution comprising the first antibody and the second antibody.

Preferably, the first antigen is an endogenous antigen, and the method further comprises repeating steps a-d with a second SAM to obtain a second mass spectrum using a control antigen and analyzing the second mass spectrum to determine the concentration of the first antigen in the sample, the control antigen comprising a portion of the first antigen. The second antigen may be a derivative of the first antigen or the second antigen comprises a tag bound to the first antigen. The second antigen may be a truncated portion of the first antigen. The first antigen may be, for example, cystatin C (CysC) and the second antigen is a truncated portion of CysC. The first antibody may be an antibody against a protein selected from the group consisting of cystatin C (CysC), hemoglobin (hem), human albumin serum (HSA) and transferrin. The receptor protein may comprise an Fc binding protein, such as protein A, protein G or a combination thereof. The first antigen may be hemoglobin A and the first antibody may be an antibody against hemoglobin adapted to bind to at least one of the alpha or beta subunit portions of the hemoglobin A.

In yet another exemplary embodiment, an immunoassay method of detecting an antigen in a sample is provided using a first antibody bound to a self-assembled monolayer (SAM), the method comprising one or more of the steps of: (a) contacting a SAM comprising an oligo(ethylene glycol) alkanethiol and a capture ligand with a receptor protein in a manner effective to bind the receptor protein to the capture ligand; (b) contacting the receptor protein bound to the SAM with the first antibody in a manner effective to bind the first antibody to the receptor protein to form a bound first antibody; (c) contacting the bound first antibody with the sample in a manner effective to bind a first antigen in the sample to the first antibody; (d) performing matrix assisted laser desorption ionization mass spectrometry on the SAM to obtain a mass spectrum including at least one mass-to-charge peak characteristic of the bound first antibody; and/or (d) analyzing the mass spectrum to determine if the first antigen attached to the bound first antibody.

Another method provides for determining the concentration of a first antigen in a sample using a first antibody bound to a self-assembled monolayer (SAM), and comprises the steps of: (a) preparing a plurality of test solutions comprising a fraction of the sample and a predetermined concentration of a control antigen; (b) contacting a plurality of SAMs each including a receptor protein with the first antibody in a manner effective to bind the first antibody to the receptor protein to form a bound first antibody bound to the SAMs; (c) contacting each test solution with one of the plurality of SAMs including the receptor protein and the first antibody in a manner effective to competitively bind the first antigen or the control antigen in each test solution to the bound first antibody; (d) performing matrix assisted laser desorption ionization mass spectrometry (MALDI-MS) on each of the SAMs after contacting each test solution with the SAM, and obtaining a mass spectrum from the MALDI-MS from each test solution including at least one mass-to-charge peak characteristic of the bound first antibody; and/or (e) analyzing the mass spectra to determine the concentration of the first antigen in the sample.

An immunoassay method of simultaneously detecting multiple antigens in a sample using a first self-assembled monolayer (SAM) including a first antibody and a second SAM including a second antibody, the method comprising the steps of: (a) contacting a SAM molecule including a first receptor protein with the first antibody in a manner effective to bind the first antibody to the first receptor protein to form the first SAM; (b) contacting a SAM molecule including a second receptor protein with the second antibody in a manner effective to bind the second antibody to the second receptor protein to form the second SAM, the second receptor protein being the same or different from the first receptor protein and the second antibody being selected to bind to a different antigen than the first antibody; (c) contacting the first SAM and the second SAM with the sample in a manner effective to bind a first antigen in the sample to the first antibody and a second antigen in the sample to the second antibody; (d) ionizing and ablating the first SAM and the second SAM, and any of the first antigen and the second antigen bound thereto, and obtaining a mass spectrum including mass-to-charge peaks characteristic of the first antibody bound to the first SAM and the second antibody bound to the second SAM; and/or (e) analyzing the mass spectrum to determine if the first antigen and the second antigen are present in the sample. Multiple antibodies may be bound to the SAM from the same solution (e.g., a mixture of antibodies) onto the same SAM surface. Multiple antigens may be subsequently bound to the SAM presenting two or more antibodies on a single SAM.

A method of diagnosing multiple sclerosis is provided using an antibody to cystatin C (cysC) bound to a self-assembled monolayer (SAM), the method comprising the steps of: (a) contacting a SAM including a receptor protein with the cysC antibody in a manner effective to bind the cysC antibody to the receptor protein to form a bound cysC antibody; (b) contacting the bound cysC antibody with the sample in a manner effective to bind a cysC antigen in the sample to the cysC antibody; (c) ionizing and ablating the SAM, the bound cysC antibody and any of the cysC antigen bound to the bound cysC antibody and obtaining a mass spectrum including mass-to-charge peaks characteristic of the bound cysC antibody; the uncleaved cysC antigen, and any cleaved cysC antigen; and/or (d) analyzing the mass spectrum to determine the ratio of the uncleaved and cleaved cysC antigen attached to the bound cysC antibody.

A method of diagnosing thalassemia is provided using an antibody to hemoglobin A (hem) bound to a self-assembled monolayer (SAM), the method comprising the steps of: (a) contacting a SAM including a receptor protein with the hem antibody in a manner effective to bind the hem antibody to the receptor protein to form a bound hem antibody; (b) contacting the bound hem antibody with the sample in a manner effective to bind a hem antigen in the sample to the hem antibody; (c) ionizing and ablating the SAM, the bound hem antibody and any of the hem antigen bound to the bound hem antibody and obtaining a mass spectrum including mass-to-charge peaks characteristic of the bound hem antibody, the alpha subunits of the hem antigen attached to the bound hem antibody, and beta subunits of the hem antigen attached to the bound hem antibody; and/or (d) analyzing the mass spectrum to determine the ratio of alpha and beta subunits of the hem antigen bound to the bound hem antibody.

A method of making an immunoassay biochip is provided including a surface with a first antibody bound to a first self-assembled monolayer (SAM) overlapping with a second antibody bound to a second SAM, the method comprising the steps of: (a) contacting a portion of the biochip surface with a SAM in a manner effective to bind the SAM to the biochip surface portion, the SAM being adapted to bind to a receptor protein; (b) contacting the SAM with a receptor protein in a manner effective to bind the receptor protein to the SAM to form a bound receptor protein; (c) preparing a mixture of a first antibody and a second antibody; and (d) contacting the receptor protein bound to the SAM with the mixture of the first antibody and the second antibody in a manner effective to competitively bind the first antibody to the bound receptor protein to form a first SAM or to bind the second antibody to the bound receptor protein to form a second SAM.

A method of diagnosing a medical condition characterized by post translational modification of an antigen is also provided by using an antibody for the antigen bound to a self-assembled monolayer (SAM), the method comprising the steps of: (a) contacting a SAM including a receptor protein with the antibody in a manner effective to bind the antibody to the receptor protein to form a bound antibody; (b) contacting the bound antibody with the sample in a manner effective to bind an unmodified antigen or modified antigen in the sample to the bound antibody; (c) ionizing and ablating the SAM, the bound antibody and any of the antigen bound to the bound antibody and obtaining a mass spectrum including mass-to-charge peaks characteristic of the bound antibody; the unmodified antigen, and any modified antigen; and (d) analyzing the mass spectrum to determine the ratio of the modified and unmodified antigen attached to the bound antibody. The ratio of the modified and unmodified antigen may be correlated to diagnose the medical condition. The modification of the antigen may include at least one modification selected from the group consisting of: Acetylation, ADP-ribosylation, Amidation, Beta-methylthiolation, Biotinylation, Bromination, Carboxylation, Citrullination, C-Mannosylation, Cysteinylation, Deamination, Diacylglycerol modification, Dimethylation, FAD modification, Farnesylation, Hydroxyfarnesylation, FMN conjugation, Formylation, Geranyl-geranylation, Glycosylation, Glutathionylation, Hydroxylation, Lipidation, Lipoic acid modification, Methylation, Myristolation, S-nitrosylation, palmitoylation, phosphorylation, Pyruvic acid modification, Stearoylation, Sulfation, SUMOylation, Trimethylation, Ubiquitination and proteolytic cleavage.

Biochips and other articles of manufacture comprising a SAM adapted to immobilize an antibody or other protein are also provided. A protein chip may be formed as an array of regions containing immobilized protein(s), optionally separated by regions containing no protein or immobilized protein at a much lower density. For example, a protein chip may be prepared by applying SAMs containing a moiety selected to selectively bind a desired protein and/or SAMs containing a mixture of such moieties. Other regions of the protein biochip surface may include a moiety that produces an inert surface on regions of the surface that are to have proteins attached or are intended to have proteins at a higher density. Inert SAMs include those containing moieties which are terminated in short oligomers of the ethylene glycol group (OCH₂CH₂)ₙOH where n is any integer, but preferably 3-6, or a moiety which is terminated in a group having multiple hydroxyl groups, such as mannitol. The remaining regions could be left uncovered, or could be covered with SAMs that are inert. The rapid kinetics of binding and covalent immobilization of protein moieties to a surface, by way of a covalent reaction with a reactant ligand, facilitates the use of spotting to deposit proteins onto the surfaces. The arrays can be rinsed to remove all but the specifically immobilized fusion polypeptides. The biochip may include one or more different SAMs on a single surface. For example, a biochip surface may include two or more types of SAMs selected from the group consisting of a first SAM comprising a bound first protein, a second SAM comprising a second protein, a third SAM comprising a bound antibody and a fourth SAM comprising a bound antigen.

In one aspect, a biochip may comprise a surface-bound self-assembled monolayer comprising oligo(ethylene glycol) alkanethiol bound to a receptor protein distal to the surface and a first antibody bound to the receptor protein. For example, the first antibody may be an antibody for an antigen selected from the group consisting of: cysC, hem, HSA and transferrin. Optionally, the receptor protein comprises at least one Fc binding protein. Accordingly, a SAM may be described according to any of the following formulae:

Surf-S-Y-C-Z (1)

Surf-S-Y-C-Z-Ab (2)

Surf-S-Y-Z-An (3)

where “Surf” designates the surface to which the SAM is bound (e.g., a biochip surface), “-S-Y-” refers to a chain portion of the SAM, “-C-” refers to a capture ligand or capture tag, “-Z-” refers to a receptor protein bound to the capture ligand or capture tag, “-Ab” refers to an antibody bound to the receptor protein on the SAM, and “-An” refers to an antigen bound to the receptor protein on the SAM. Alternatively, the biochip may include a protein such as an antibody bound to a SAM without a receptor protein. For example, a SAM comprising a maleimide moiety may be bound to IDA, and the IDA may be complexed with a cationic transition metal such as cobalt in the 2+ or 3+ oxidation state. The SAM-bound cation may be selected to bind an antibody such as an antibody for albumin to the SAM to provide a surface that selectively binds an antigen such as albumin when the SAM is contacted with a complex sample obtained from a living subject (e.g., a clinical sample such as blood, urine or cerebral spinal fluid). Accordingly, a SAM may be described according to any of the following formulae:

Surf-S-Y-C-D (4a)

Surf-S-Y-D-C (4b)

Surf-S-Y-C-D-Ab (5a)

Surf-S-Y-D-C-Ab (5b)

Surf-S-Y-C-D-An (6a)

Surf-S-Y-D-C-An (6b)

where “Surf” is defined above, the chain portion (“-Y-”) includes a maleimide terminated SAM molecule extending from an alkanethiolate portion bound to the surface and an ethylene glycol portion bound to the alkanethiolate portion and the maleimide portion, the capture ligand (“-C-”) includes, for example, IDA complexed with a moiety (“-D”) such as an ionized transition metal (Co²⁺ or Co³⁺) that binds to an antibody (-Ab) or antigen (-An), or other protein. The moiety (“-D”) may also be a transition metal cation (e.g., Ni²⁺) selected to bind or coordinate with the capture ligand (e.g., triazacyclononane) that may bind a protein.

The chain portion of the SAM preferably includes an alkane thiolate portion and an ethylene glycol portion and may be configured to minimize non-specific absorption or adsorption to the SAM surface, and to bind and orient molecules bound to the SAM in a manner the provides a desired activity to the bound molecules. The chain portion is preferably an alkanethiol terminated in the penta(ethylene glycol) portion having 3-6 repeating ethylene glycol monomer units. The loading of the antibody, antigen, capture ligand, capture tag or receptor protein may be varied. Preferably, the loading of a protein is less than 100% of the SAM surface area, more preferably about 50% or less, and most preferably about 25% or less, including loading levels of about 0.5%-25%, 0.5%-15%, 0.5%-10%, 0.5%-5%, and 0.5%-3%.

A biochip may comprise a surface portion including a mixture of: (a) a first surface-bound self-assembled monolayer comprising oligo(ethylene glycol) alkanethiol bound to a receptor protein and a first antibody bound to the receptor protein; and/or (b) a second surface-bound self-assembled monolayer comprising oligo(ethylene glycol) alkanethiol bound to a receptor protein and a second antibody bound to the receptor protein. The first antibody and the second antibody may be antibodies for an antigen selected from the group consisting of: cysC, hem, HSA and transferrin.

### EXAMPLES

The following examples describe the uses for ligand-modified self-assembled monolayers. Mass spectrometric methods offer a significant benefit over optical methods for label-free detection, including surface plasmon resonance spectroscopy. Because optical methods measure changes in the refractive index of the medium near the biosensor surface, they do not discriminate between the intended analyte and species that contribute to the background signal. SAMDI, and other methods that use mass spectrometry, provide the masses of the species interacting with the sensor surface and therefore can more efficiently identify, and quantitate, the signal for the intended analyte even when there are significant levels of background species bound to the sensor surface. Further, the mass filtering that is inherent to mass spectrometry methods permits multiple assays to be performed simultaneously. Four antigens in serum were analyzed with an immunosensor that presented a mixture of antibodies specific to each analyte. The same experiment would be difficult to perform with fluorescently-labeled reagents because of spectral overlap of the fluorophores, and would be essentially impossible to perform with radiolabeled reagents.

Preferably, the SAM is adhered to the surface of a biochip. The biochip may comprise self-assembled monolayers of alkanethiolates on a suitable metal surface (SAMs). The synthesis of SAMs is well known in the art (See, for example, U.S. published applications 20020119305 and 20020119054).

### Example 1

### Preparation of Monolayers

Monolayers were prepared as reported elsewhere (e.g., Houseman, B. T.; Gawalt, E. S.; Mrksich, M. Langmuir 2003, 19, 1522-1531). Briefly, 60 nm of titanium followed by 220 nm of gold were evaporated by electron beam onto a microscope cover glass (cat. 12-543-D) from Fisher Scientific (Pittsburgh, Pa.). Self assembled monolayers were generated by immersion of the metallized slides in an ethanolic solution containing an asymmetric disulfide derived from tri(ethylene glycol)- and maleimide-terminated alkanethiolates and a symmetric disulfide derived from the tri(ethylene glycol)-terminated alkanethiolate for 12 hours (1 mM, ratios range from 1:9 to 1:49). The triazacyclononane (aza) or nitrilotriacetic acid (NTA) ligands were immobilized by treating the monolayer with the parent thiol-substituted reagents for one hour (1 mM, phosphate buffered saline (PBS), pH 7.2). The monolayers were treated with an aqueous solution of NiSO₄ for five minutes to generate the Ni²⁺ complex (1 mM, PBS, pH 7.2).

For immunoassays, monolayers were cut into square pieces measuring 4 mm² and treated for five minutes with His-tagged Protein A (48 kDa cat. 6510-00) (Akerstrom, B.; Bjorck, L. J Biol Chem 1986, 261, 10240-10247; Akerstrom, B.; Brodin, T.; Reis, K.; Bjorck, L. J Immunol 1985, 135, 2589-2592) or Protein G (27 kDa, cat. 6500-10) (Goding, J. W J Immunol Methods 1978, 20, 241-253; Kronvall, G.; Seal, U.S.; Finstad, J.; Williams, R. C., Jr. J Immunol 1970, 104, 140-147) (10 uL, ˜1-10 μM, PBS, pH 7.2) from Biovision Inc. (Mountain View, Calif., USA). The resulting monolayers were then treated with the IgG-antibodies for five minutes (10 uL, ˜5 uM, PBS, pH 7.2). Antibodies to human albumin (cat. A80-129A), human hemoglobin (cat. A80-134A), and human transferrin (cat. A80-128A) were purchased from Bethyl Laboratories Inc. (Montgomery, Tex., USA). Anti-human cystatin C (cat. A0451) was purchased from Dako A/S (Carpinteria, Calif., USA). Chips were washed under a stream of water and dried with a stream of nitrogen after each step. Protein A or G surfaces could be stored several days at 4° C. prior to use.

### Example 2

### Antibody/Antigen Assays

Serum and CSF samples were diluted 1:1 with PBS (pH 7.2), spotted onto the immunosensor (in a volume of 5 uL) and incubated for five minutes. Serum (cat. D119-00-0050) was obtained from Rockland Immunochemicals (Gilbertsville, Pa., USA) and plasma (cat. CCN-10) was obtained from Precision BioLogic (Dartmouth, Nova Scotia, Canada). CSF samples, collected under an IRB approved protocol, were generously donated by Dr. Anthony Reder (University of Chicago, Department of Neurology). Quantitation of endogenous human cystatin C (ₕcysC, 13.4 kDa) in CSF was performed by comparison of peak areas to an internal control. Cystatin C obtained from human urine (_{trunc}cysC, 12.5 kDa, cat. 240896) was purchased from EMD Biosciences (San Diego, Calif., USA), diluted with PBS, and spiked into the CSF (at various concentrations) prior to application on the immunosensor. Chips were washed under a stream of water (˜10 sec) after sample incubation and dried under a stream of nitrogen.

### Example 3

### Mass Spectrometry

Either sinapinic acid (SA) or α-cyano-4-hydroxycinnamic acid (CHCA) at 5-7.5 mg/ml (in acetone, 0.3 uL manually spotted) was used to facilitate protein desorption and ionization from the monolayer surface. Mass analysis was performed on a Voyager DE-PRO Biospectrometry mass spectrometer from Applied Biosystems (Framingham, Mass., USA). The instrument has a 337-nm nitrogen laser and typical experimental parameters include: linear mode of operation, manual acquisition, positive ion polarity, 750 nsec delayed extraction, 25000 VDC acceleration voltage, 97.5% grid voltage and 0.3% guide wire. Spectra obtained are the average of ˜2000 shots. Data were smoothed with a Gaussian routine. To quantitate ₕcysC, the spectra were baseline corrected prior to integration of peak areas.

### Example 4

### Protein-Protein Interaction Mapping

Recent work has demonstrated the feasibility of using SAMDI to screen protein-protein interactions (FIG. 1A). This work examined protein interactions in Shewanella oneidensis. Thirty proteins were expressed in the his-tagged form—that is, each protein had a oligohistidine sequence at the N-terminus—and a fraction of each expressed protein was treated with TEV protease to generate a corresponding set of proteins that were non-tagged. In this way, in separate experiments, 2 μL of each his-tagged protein was mixed with 2 μL of each non-tagged protein, to give a total of 900 combinations (this mixing is performed in microtitre plates). Within this set, each pair of proteins is represented twice, since either the first or the second protein may carry the his-tag. To determine whether the two proteins interact, a microliter of the solution was applied to a self-assembled monolayer that presents aza-Ni²⁺, an analogue of the NTA-Ni²⁺ complex which is well known to interact with the his-tags to give a selective and kinetically stable link (FIG. 1A). In this way, the his-tagged protein is immobilized to the monolayer, and if this protein interacts with the non-tagged protein, both proteins become immobilized to the monolayer. After a 10 minute incubation to allow the his-tagged protein to immobilize, the monolayer was rapidly rinsed, a matrix applied, and the surface analyzed with MALDI-TOF mass spectrometry to observe the proteins.

The his-tagged protein was observed in each spectrum—that is, SAMDI can identify proteins that are immobilized to the monolayer, over a mass range of 5,000 to 200,000 Daltons (FIG. 1B), including mass ranges of about 5,000 to 100,000, 5,000 to 150,000 and 5,000 to 175,000 Daltons. FIG. 1C shows a spectrum for a pair of proteins that interact. The top spectrum shows two peaks corresponding to each of the proteins, with the higher mass protein carrying the his-tag. The lower spectrum corresponds to the interaction when the other protein is his-tagged. This characteristic ‘doublet of doublets’ pattern is a fingerprint for the interaction (see also FIG. 1 D,E). There is also the possibility that the his-tag, or the surface, can interfere with the interaction. The several interactions that were observed in only one of the two orientations may provide evidence for this possibility (see those boxes shaded purple in FIG. 1F). Where the proteins had masses that were smaller than the predicted masses based on the gene sequence (the green boxes in FIG. 1F), this was likely due to proteolysis or mutations that introduce stop codons. This observation points to the significant benefits that SAMDI brings to quality control—the mass resolution of this technique permits a verification that the proper bait protein is presented on the chip and can separate signals for the intended analyte from background analyte, when enough information is available to anticipate the interaction. This label-free and general route to observing proteins in a format that is compatible with biochips is remarkable and enables a robust approach to screening interactions.

### Example 5

### High Throughput Protein-Protein Interaction Mapping

The following procedure was used repeatedly to screen 96 interactions: this protocol represents a conservative estimate of the resources required to screen a number of interactions. In this experiment, the interaction of a single his-tagged protein with each of 96 non-tagged proteins was screened. A 96-well plate containing the non-tagged proteins was used with an automated pippetting station to transfer 2 μL of each protein from the source plate to the analogous well in the target plate. The pipetting station was used to transfer 2 μL of the same his-tagged protein to each of the 96 wells in the target plate. The final volume of 4 μL is greater than is needed in the assay, but is chosen to prevent significant evaporation of the solution during incubation. Other methods can be employed to minimize the evaporation and in turn to permit the use of much smaller volumes of protein-containing solutions. A low retention 96-well PCR plates with tapered ends was used to handle these volumes of solution. The target plates were typically incubated for 30 min to provide sufficient time for the interactions to reach equilibrium. For the biochip, a plate was used that is compatible with the software and staging mechanism in the MALDI-TOF mass spectrometer (a Voyager DE-PRO model). The commercial plates have a 10 by 10 array of grooves etched into the metal plate. In our application, a glass slide measuring 1.5×1.5 inches was used, onto which gold was deposited through a rigid brass stencil in an array of 10×10 circles. In this way, the sizes and positions of the 100 spots on the plate are nearly identical to those in the commercial plates, permitting an automation in acquiring the data. In practice, a micropipette was used to manually transfer 2 μL of each protein solution from the target plate to a spot on the SAMDI plate. Other liquid transfer methods may be employed that reduce the volumes of sample, and that can greatly increase the density of distinct samples that are arrayed onto a substrate. The array of droplets was allowed to stand at room temp for 30 min, in humidified chambers, the droplets were quickly rinsed off the surface, matrix applied and the mass spectra acquired.

The methods described herein may be modified to increase the throughput of the approach. One of these modifications includes the mixing of each his-tagged protein with a combination of non-tagged proteins. Because the probability of an interaction is low (in the case where fewer than approximately 100 proteins are used), there is little chance that an interaction will be masked by a stronger competing interaction. Further, because the mass of each protein is known, it is straightforward to assign the protein-protein interaction. This approach can conservatively be expected to increase the throughput by one to two orders of magnitude.

### Example 6

### Assembly of Surfaces for Immunoassays

SAMDI was used to characterize the assembly of two model immunosensors where either Protein A or Protein G receptor proteins were used to immobilize cystatin C (cysC) antibody (FIG. 2). A solution of either the 48 kDa ₕᵢₛProtein A or 27 kDa ₕᵢₛProtein G (5 μM in PBS with 30% glycerol) was incubated for 5 minutes at room temperature on the monolayer presenting the aza/Ni²⁺ group at a density of 1% (relative to the total alkanethiolate). Following the incubation, the monolayers were rinsed with water and then incubated with the antibody (5 μM in PBS) for 5 minutes at room temperature. The surface was then rinsed again with water and incubated with cysC (0.3 μM in PBS) for 5 minutes at room temperature. The monolayers were rinsed with water, dried, treated with matrix and analyzed by SAMDI mass spectrometry.

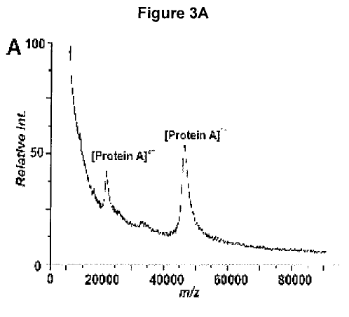

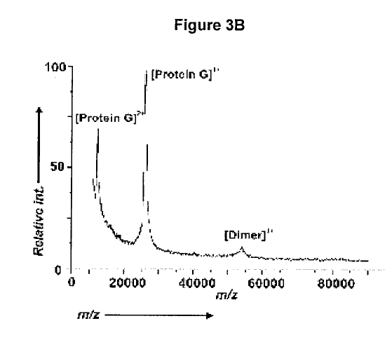

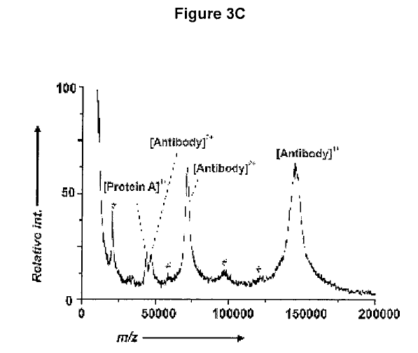

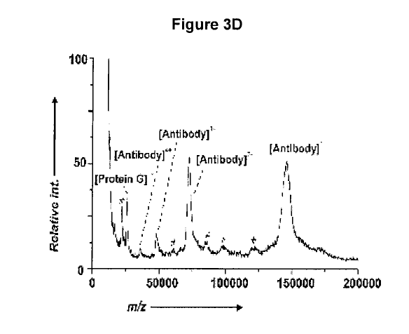

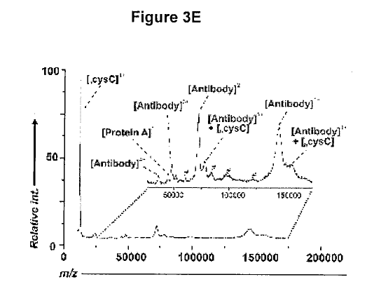

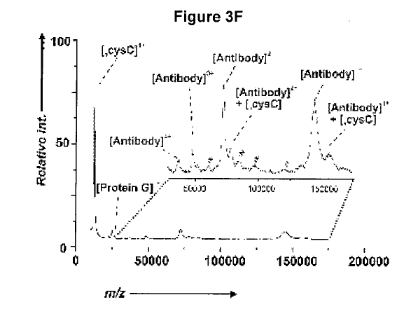

The SAMDI spectra reveal the proteins that bind to the surface in each of the steps described above (FIGS. 3A-3F). The 10,000-90,000 m/z region of the mass spectra in FIGS. 3A and 3B reveal clear peaks for Protein A and Protein G, respectively. Multiple charge states and dimeric species are typically observed for both samples. Control experiments conducted in the presence of 300 mM imidazole—which disrupts interaction of the histidine tag with the aza-Ni²⁺ complex—resulted in minimal retention of the bound protein (data not shown). The antibodies efficiently bound to the Protein A (or G) on the monolayers (FIGS. 3C and 3D). Multiple charge states were identified, corresponding to the antibody across the 15,000-200,000 m/z region of the spectrum including an intense peaks associated with the singly and doubly charged ions at 150 kDa and 75 kDa, respectively.

Antibodies that were immobilized as described above efficiently captured the cysC antigen present in the sample (FIGS. 3E and 3F). Following binding of the antigen to the immobilized antibody, SAMDI revealed peaks for each of the three proteins on the surface—Protein A/G, antibody and antigen—although, the intensity of the peak representing the receptor protein decreased in the presence of the other proteins. This difference may be due to more efficient desorption and ionization of the antibody and antigen from the Protein A/G. Interestingly, peaks corresponding to the cysC in a non-covalent complex with the antibody survived the ionization process. However, these peaks alone do not distinguish whether the antigen is still bound to the variable Fv binding region of the antibody or present as a loosely associated gas phase product (see inset, FIGS. 3E and 3F). These examples demonstrate that SAMDI, in addition to its value in performing diagnostic assays (see below), is useful for verifying the integrity of the immunosensor. Measurement of constituently loaded proteins in parallel offers a significant degree of quality control in the development and application of the immunosensor, especially in the presence of complex proteinaceous mixtures, such as serum.

### Example 7

### Model Immunoassays

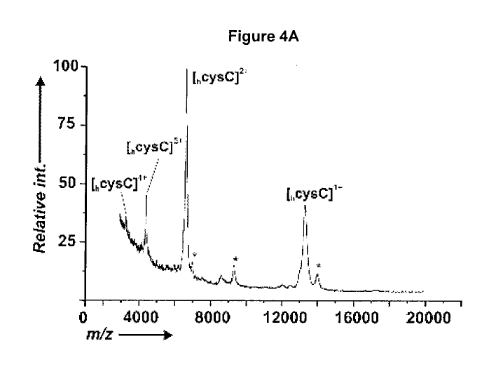

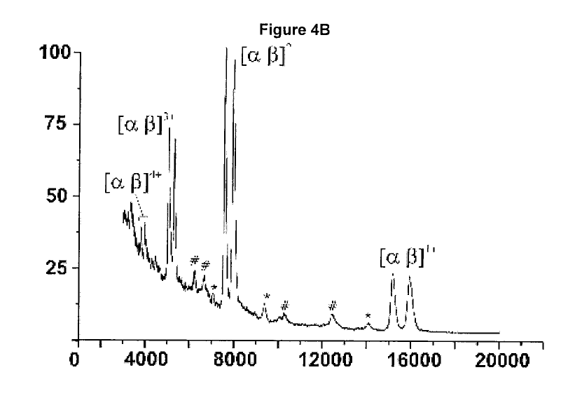

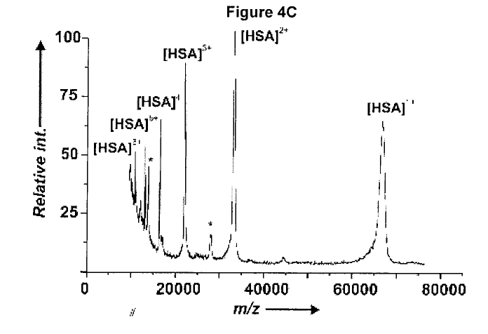

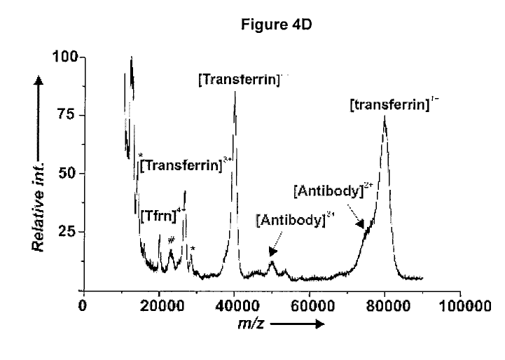

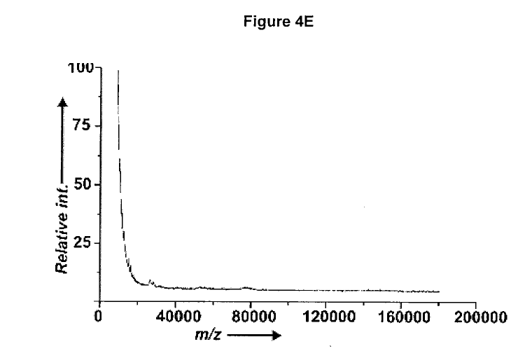

Although this immunosensor strategy requires multiple steps to prepare the surface for an assay, it has the advantage that it only requires a single primary antibody to detect a designated antigen and therefore can readily be applied to a broad range of assays. In one series of experiments, several antigens from human serum were detected by SAMDI (FIGS. 4A-4E) using commercially available antibodies raised against the proteins cystatin C (cysC), hemoglobin (hem), human serum albumin (HSA), and transferrin (transferrin). In each case, the antibodies were immobilized by way of Protein G as described in the previous section. For each assay, human serum was diluted with an equal volume of phosphate buffered saline (PBS), incubated for 5 minutes on the immunosensor, washed with water, and dried under a stream of nitrogen prior to application of the CHCA matrix. The spectra in FIGS. 4A-4E show that cysC, hem, HSA, and transferrin were efficiently and selectively bound to the surface from serum and detected by SAMDI mass spectrometry.

The SAMDI methods may be used to analyze protein complexes such as hemoglobin A. In the case of the hem immunosensor (FIG. 4B), both subunits of hemoglobin A—comprised of two α (15.1 kDa each) and two β (15.9 kDa each) subunits—were detected in parallel which indicates the polyclonal antibody was specific for the capture of the individual subunits or the entire non-covalent tetramer that then dissociated during the ionization process. The SAM may include an antibody to hemoglobin A, or its subunits (alpha or beta). In one example, a SAM may selectively bind a protein complex, or portion thereof, such as the alpha and/or beta portions of hemoglobin A. SAMDI analysis of the resulting surface may be used to identify the presence of the protein complex or subunits thereof in a complex endogenous clinically-derived fluid sample.

The immunoassays presented in FIGS. 4A-4E were specific for the intended analyte, showing that the surfaces are resistant to non-specific adsorption from other proteins in the serum samples. To further ascertain the role of non-specific binding of serum proteins, a monolayer presenting only tri(ethylene glycol) groups (in the absence of Ni²⁺, receptor proteins, or antibodies) was incubated with the human serum sample and only a minimal contribution of any single serum component to the base monolayer was detected, with none of these components reproducibly present in the antibody loaded assays (FIG. 4E). In several of the assays presented in FIGS. 4A-4E, low intensity unidentified peaks were observed (typically <5% peak intensity relative to the base peak) although for the transferrin immunosensor (FIG. 4D) there was a dominant cluster of peaks present at ˜12 kDa. These species may derive from protein components that were present during the immobilization of the antibody and appear to have affinity for the Protein A/G surface since these same components were observed prior to treatment of the substrate with sample (e.g., FIGS. 3C & 3D vs. 3E & 3F for cysC).

In this work both sinapinic acid (SA, 5 mg/mL in acetone) and α-cyano-4-hydroxycinnamic acid (CHCA, 7.5 mg/mL) were used as the energy absorbing matrices in this work. In each case, approximately 0.3 μL of matrix was spotted by hand with a micropipette and allowed to diffuse across the monolayer. Signals were typically observed across the entire surface, but included a variation of signal intensity and some concentration of the non-covalently bound components at the matrix frontal edge. Typically, SA provided greater S/N for the m/z>50,000 portion of the spectrum, which offered a more pronounced signal for the antibodies. In these instances, a greater surface area was required for sampling because higher laser intensities were necessary to obtain the spectra which follow previous observations. The CHCA matrix permitted the use of lower laser intensities and therefore provided prolonged sampling across a given area of the surface, while yielding good peak intensities for the m/z<50,000 portion of the spectrum. Signal resulting from the loaded antibodies was sometimes suppressed with CHCA while that of Protein A/G molecule and antigen could still be observed (see FIG. 4C for HSA vs. FIG. 4D for transferrin). This effect may be a response to the high density of antigen loaded across the surface that may interfere with desorption and ionization of the antibody. Alternatively, this observation could result from localized concentration of antigens to regions of the immunosensor that were manually sampled.

### Example 8

### Multi-Analyte Format

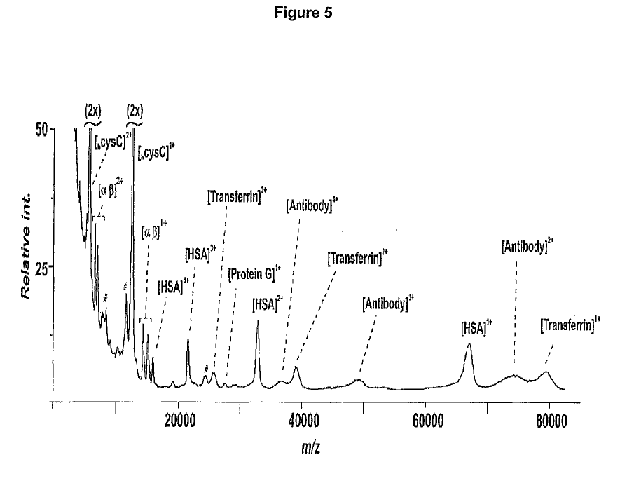

A benefit of mass spectrometry is that the antibodies can be applied as a mixture to the monolayer and need not be arrayed in non-overlapping regions of the substrate to resolve the binding of each antigen—as is the case when fluorescence measurements are used to interrogate the chip. To demonstrate this capability with SAMDI, a single monolayer that presented the four antibodies described above was prepared and used to identify analytes from human serum. The four antibodies were mixed in a ratio of 1:2:2:4 for cysC, hem, HSA, and transferrin, respectively, and applied to a monolayer presenting Protein G. The substrate was treated with serum for 5 minutes, rinsed, treated with matrix and analyzed by SAMDI. The 5,000-90,000 m/z region of the SAMDI spectrum shows peaks that correspond to Protein G, the antibodies, and each of the antigens in multiple charge states (FIG. 5). Nonequimolar amounts of the antibodies were required in order to adequately observe peaks for each of the antigens. The differences in peak intensity presumably reflect a combination of the affinity of the antibody for the antigen and the ionization efficiency of the antigen. Application of SAMDI for evaluation of multiple proteins offers a significant advantage for clinical diagnostics by significantly reducing the time and sample consumption per assay.

### Example 9

### Quantitation of Antigens

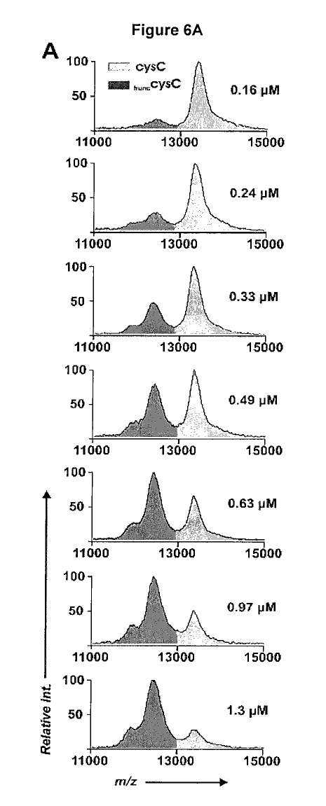

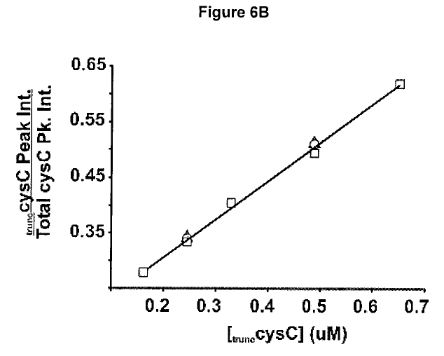

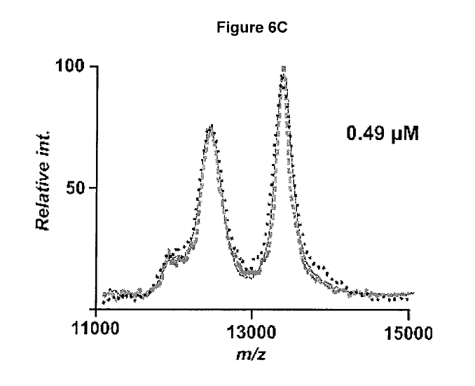

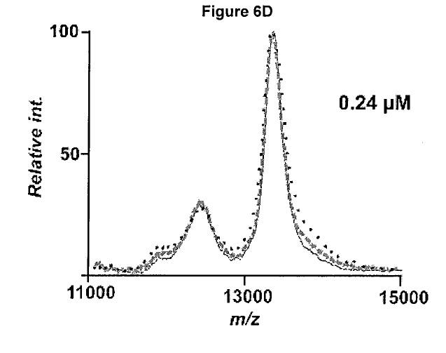

The common strategy for obtaining quantitative data in mass spectrometry experiments relies on the addition of a known standard to a sample, and comparing the intensity (or peak area) of the analyte to that of the standard. This strategy was used for the detection of cysC in human cerebral spinal fluid (CSF) (FIG. 6). A truncated form of cysC (_{trunc}cysC, 12.5 kDa), obtained from human urine, was used as an internal standard. Serial dilutions of the _{trunc}cysC were added directly to CSF samples (160-1300 nM, final concentration) and each of the samples was then applied to the immunosensor. SAMDI spectra in the mass range of 11000-15000 m/z show competitive binding between the _{trunc}cysC and the endogenous cysC (FIG. 6A). At higher concentrations of _{trunc}cysC, the spectra revealed a shoulder at ˜11 kDa (in addition to the dominant peak at 12.5 kDa) that were treated as part of the _{trunc} cysC peak area.

Clinical diagnostic assays based on the enzyme-linked immunosorbent assay (ELISA) provide a linear response across a large concentration range, and provide standards of reproducibility with coefficients of variation (CV) typically less than 10%, and low detection limits (˜1 pg/ml) for a diverse array of antigens. The chart in FIG. 6B shows the calculated ratio of the _{trunc}cysC peak areas to the total cystatin C area (from FIG. 6A) that span the reference range for cystatin C in CSF fluid. A linear increase in _{trunc}cysC area was observed with a corresponding increase in _{trunc}cysC concentration. Using samples of _{trunc}cysC at concentrations of 0.24 μM and 0.49 μM, SAMDI spectra were obtained on different days, using monolayers having a range of density of the aza/Ni⁺² ligand (0.5%, 1.0%, % 4%) (FIGS. 6C and 6D). The similar peak shapes and ratios observed over the course of these experiments highlights the good reproducibility of this immunoassay. The concentration of endogenous cysC, based on the calculated ratios of the nine peak ratios presented in FIG. 6B, was determined to be 0.459±0.030 μM with a coefficient of variation (CV) of 6.5%. In principle, the control antigen should closely mimic the endogenous antigen in its affinity for the antibody and ionization properties. This similarity is apparent for the example presented in FIG. 6 since there is a direct competitive relationship between the control and the endogenous antigen. It may be possible to identify an appropriate internal standard by selecting a metabolic derivative of the endogenous antigen or by preparing a covalently tagged version of the antigen that does not interfere with the affinity for the antibody. The lower limits for detection with this assay will ultimately depend on the affinity of the antibody for the antigen and possibly by the intensity of peaks for background species. Signal was still observed for cystatin C above background (data not shown) even after a cystatin C dilution of about 200-fold, translating into a lower limit for detection of about 30 ng/ml.

### Example 10

### Detection of Post-Translational Modifications in CSF

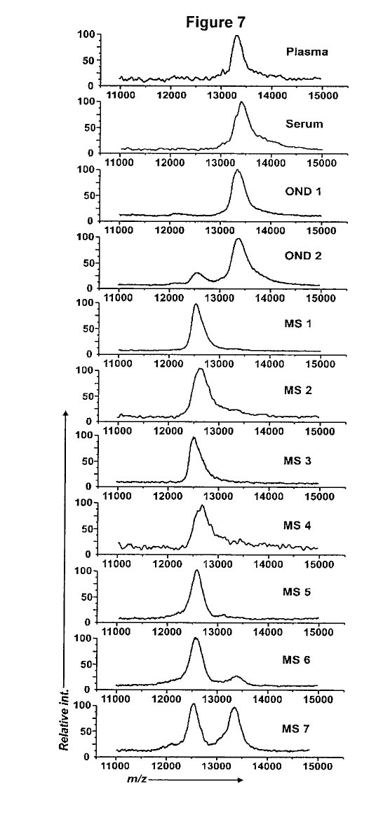

A recent study has suggested that a proteolytic fragment of cysC may serve as a marker for multiple sclerosis.⁵⁴ Nath and coworkers used SELDI-TOF to identify a 12.5 kDa form of cysC in the cerebral spinal fluid (CSF) of multiple sclerosis patients. This form of cysC has a mass that is 900 Da lower than that for the parent 13.4 kDa form that is present in CSF from healthy individuals. In an experiment that included 27 diseased patients and 27 control patients, presence of the proteolytically derived fragment of cysC was found to be 100% specific for patients with multiple sclerosis versus other neurological diseases. Several CSF samples were evaluated to measure the relative amounts of the 12.5 kDa and 13.4 kDa forms of the cysC protein in clinical samples isolated from patients with and without multiple sclerosis. Seven CSF fluid samples from patients having multiple sclerosis were analyzed and compared to two samples from patients with other neurological diseases, as well as serum and plasma samples from disease-free patients (FIG. 7). The 11000-15000 m/z region of each SAMDI spectrum revealed that the truncated form of the cysC was present in all patients that had multiple sclerosis, while in two cases the normal 13.3 kDa form of the protein was present at ˜10% and ˜50% of the total cysC captured. In the control samples, the serum, the plasma, and one of the CSF samples showed only the unmodified 13.3 kDa form of cysC, while in one other CSF sample there was a minor amount (˜15%) of the observed cysC in the 12.5 kDa form. This species may derive from long-term storage conditions where exposure to temperatures >−20° C. results in a similar truncation as observed in patients with multiple sclerosis (Del Boccio, P.; Pieragostino, D.; Lugaresi, A.; Di Ioia, M.; Pavone, B.; Travaglini, D.; D'Aguanno, S.; Bernardini, S.; Sacchetta, P.; Federici, G.; Di Ilio, C.; Gambi, D.; Urbani, A. Ann Neurol 2006; Hansson, S. F.; Hviid Simonsen, A.; Zetterberg, H.; Andersen, O.; Haghighi, S.; Fagerberg, I.; Andreasson, U.; Westman-Brinkmalm, A.; Wallin, A.; Ruetschi, U.; Blennow, K. Ann Neurol 2006; Nakashima, I.; Fujinoki, M.; Fujihara, K.; Kawamura, T.; Nishimura, T.; Nakamura, M.; Itoyama, Y. Ann Neurol 2006). The effects of storage conditions were not evaluated as part of this study but this example does establish that SAMDI can be applied to the analysis of post-translationally derived markers in actual clinical samples.

Mass spectrometric methods are particularly useful for the characterization of proteins that contain metabolically-derived post-translational modifications or protein variants associated with gene families (or single nucleotide polymorphisms). This capability was highlighted by this invention for the identification of a truncated form of cystatin C in patients with multiple sclerosis and also in the discrimination between the α (15.1 kDa) and β (15.9 kDa) subunits of hemoglobin A. This capability was further demonstrated for α and β thalassemia, which are inherited diseases of red blood cells that manifests in a deficiency of the respective α and β chains in hemoglobin. The extent of the disease is dependent upon the number of loci that are affected. The SAMDI immunosensor may offer a simple method to quantitatively compare the ratio of these two subunits (without the use of an internal standard) and thus provide a prognosis for disease.

### Example 11

### Antibody Detection of Albumin

This example describes the use of a SAM having an aminodiacetic acid (IDA) capture ligand that can be bound directly to a maleimide terminated SAM surface via the free thiol without using a receptor protein. The IDA capture ligand may have the following formula:

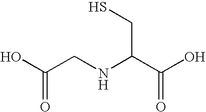

The IDA ligand bound to the maleimide terminated SAM will coordinate transition metals. For example, the transition metal cation such as Co²⁺, can subsequently immobilize an antibody directly to the SAM surface via a histidine rich region that is common in antibodies (particularly IgG antibodies). The Co²⁺ may be oxidized Co²⁺ to Co³⁺, with a <0.1% peroxide solution, forming an inert complex with antibodies to bind antibodies to the SAM. The SAM-bound antibody may be retained when the SAM is contacted with a solution comprising histidine rich proteins. Accordingly, any exchange of the SAM-bound antibodies back into solution with other histidine rich proteins in complex mixtures is reduced or avoided entirely. Thus, an antibody may be immobilized on the surface of a SAM without a receptor protein such as protein G or protein A in the SAM.

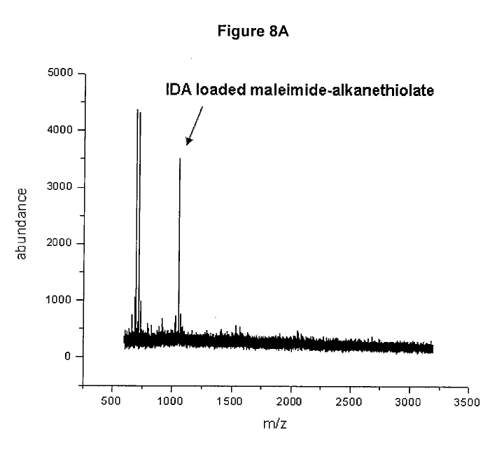

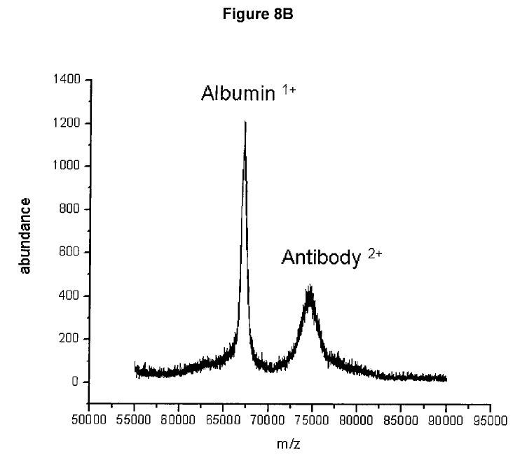

FIG. 8A and FIG. 8B are portions of SAMDI spectrum obtained from a SAM surface with about 15% of the IDA-loaded maleimide SAM surface (FIG. 8A) (1 hr), Co²⁺ (5 min), antibody for albumin (30 min), oxidized with 0.05% peroxide (3 min), and subsequently exposed to plasma to pull down albumin (30 min) to obtain the SAMDI spectrum of FIG. 8B. This example was performed in the presence of ˜45 mM EDTA (metal chelator) which shows that the antibody is in a stable complex on the surface. This IDA approach can be done with or without oxidation of Co²⁺. The Co³⁺ version is believed to be more stable than the Co²⁺, but cobalt in either the +2 or +3 oxidation state will bind and immobilize an antibody to the SAM. Optionally, the oxidation of the Co²⁺ to Co³⁺ may be performed before or after binding the antibody to the SAM. In this example, the antibody is bound to a transition metal ion attached to an IDA capture ligand that is bound to the maleimide portion of a SAM, where the SAM is free of a receptor protein (e.g., free of protein A or protein G).

Future emphasis in automation of sample handling, matrix deposition, and parallel analysis with microarrays will further expand the utility of this approach as a standardized diagnostic tool.

1. A label-free detection method of identifying a binding interaction between a first molecule and a second molecule in an endogenous sample obtained from a living subject and analyzed using a self-assembled monolayer (SAM) adapted to selectively bind the first molecule to the SAM, the method comprising the steps of: (a) contacting a first molecule with the SAM in a manner effective to bind the first molecule to the SAM, the first molecule being selected from the group consisting of: an antibody and a first protein comprising a capture tag; (b) contacting a second molecule with the first molecule in a manner effective to bind the first molecule to the second molecule, the second molecule being an antigen when the first molecule is the antibody or a second protein free of the capture tag when the first molecule is the first protein; (c) ionizing and ablating the SAM, the first molecule bound to the SAM and any of the second molecule bound to the first molecule bound to the SAM and obtaining a mass spectrum including at least one mass-to-charge peak characteristic of at least one of the first molecule and the second molecule bound to the SAM; and (d) analyzing the mass spectrum to determine if the second molecule bound to the first molecule bound to the SAM.

2. The method of claim 1, wherein the first molecule is contacted with the SAM in a manner effective to bind at least a fraction of the first molecule to the SAM followed by contacting the second molecule with the SAM in a manner effective to bind the second molecule to a fraction of the first molecule bound to the SAM.

3. The method of claim 1, wherein the first molecule and the second molecule are mixed in a solution, followed by contacting the solution with the SAM in a manner effective to permit at least a fraction of the first molecule to bind to the SAM and at least a fraction of the second molecule to bind to a fraction of the first molecule bound to the SAM.

4. The method of claim 1 for identifying a binding interaction between a first protein and a second protein using the self-assembled monolayer (SAM) adapted to selectively bind the first protein comprising the capture tag in the presence of the second protein without the capture tag, the method comprising the steps of: (a) contacting the first protein including the capture tag and the second protein free of the capture tag with the SAM in a manner effective to bind the first protein capture tag to the SAM; (b) ionizing and ablating the SAM, the first protein bound to the SAM and any of the second protein bound to the first protein bound to the SAM and obtaining a mass spectrum including at least one mass-to-charge peak characteristic of at least one of the first protein and the second protein bound to the SAM; and (c) analyzing the mass spectrum to determine if the second protein interacted with the first protein bound to the SAM.

5. The method of claim 4, further comprising the steps of: (a) contacting the second protein including the capture tag and the first protein free of the capture tag with a second SAM in a manner effective to bind the capture tag to the second SAM; (b) ionizing and ablating the second SAM, the second protein bound to the second SAM and any of the first protein bound to the second protein bound to the second SAM and obtaining a second mass spectrum with at least one mass-to-charge peak characteristic of the second protein bound to the second SAM; and (c) analyzing the second mass spectrum to characterize the interaction of the first protein with the second protein bound to the second SAM.

6. The method of claim 5, further comprising the step of characterizing the interaction of the first protein and the second protein by comparing the first mass spectrum and the second mass spectrum to characterize the binding of: (a) the second protein free of the capture tag to the first protein with the capture tag; and (b) the first protein free of the capture tag to the second protein with the capture tag.

7. The method of claim 1, wherein the method is further characterized by one or more of the following: (a) the capture tag comprises a oligohistidine sequence; (b) the SAM comprises an oligo(ethylene glycol) alkanethiol adapted to selectively bind the capture tag; (c) the SAM comprises a capture ligand that selectively binds to the capture tag; and (d) the SAM comprises a capture ligand that includes a triazacyclononane (aza) or a nitrilotriacetic acid (NTA).

8. The method of claim 4, wherein the method is further characterized by one or more of the following: (a) at least one of the first protein and the second protein is an aqueous-soluble protein; (b) at least one of the first protein and the second protein is a portion of a peptide expressed by an open reading frame of a gene; (c) at least one of the first protein and the second protein is an aqueous-soluble portion of an aqueous-insoluble peptide expressed by an open reading frame of a gene; (d) at least one of the first protein and the second protein comprises a predicted protein-protein interaction domain; and (e) the interaction of the first protein and the second protein is analyzed without SPR or detecting a fluorescent reporter group, a radioactive reporter group or a heterologous tag that is adapted to bind a fluorescent reporter.

9. The method of claim 4, wherein at least one of the first protein and the second protein has a molecular weight of about 50 kDa to about 200 kDa and the SAM has a loading of between about 0.5% and 25% of the first protein.

10. The method of claim 1, where the SAM is a self-assembled monolayer (SAM) on a biochip and the ionization and ablating of the SAM and obtaining the mass spectrum are performed using matrix-assisted laser desorption/ionization and time of flight mass spectrometry (SAMDI) comprising the steps of: (a) mixing a first molecule including a capture tag and a second molecule free of the capture tag to form a first solution; (b) contacting the first solution with a first SAM in a manner effective to bind the capture tag to the first SAM, the first SAM comprising a capture ligand that selectively binds to the capture tag; and (c) analyzing the first SAM comprising the bound first molecule and any of the second molecule bound to the first molecule bound to the SAM by SAMDI.

11. The method of claim 4, where the first protein has a mass of about 5,000 to 200,000 Daltons.

12. The method of claim 4, where the SAM is a capture-ligand-terminated oligo(ethylene glycol) alkanethiol self-assembled monolayer on a biochip, the steps of ionization and ablation of the SAM and the step of obtaining the mass spectrum are performed using matrix-assisted laser desorption/ionization and time of flight mass spectrometry (SAMDI) comprise the steps of: (a) contacting the first protein tagged with an N-terminal oligohistidine sequence capture tag and the second protein free of the N-terminal oligohistidine sequence capture tag with a first SAM in a manner effective to bind the capture tag of at least a fraction of the first protein to the first SAM; (b) removing the first protein including the capture tag that is not bound to the first SAM and the second protein that is not bound to the fraction of the first protein bound to the first SAM; (c) analyzing the first SAM by SAMDI after removing the fraction of the first protein that is not bound to the first SAM and the second protein that is not bound to the fraction of the first protein bound to the first SAM; (d) contacting the second protein tagged with an N-terminal oligohistidine sequence capture tag and the first protein free of the N-terminal oligohistidine sequence capture tag with a second SAM in a manner effective to bind the capture tag of at least a fraction of the second protein to the second SAM; (e) removing the second protein including the capture tag that is not bound to the second SAM and the first protein that is not bound to the fraction of the second protein bound to the second SAM; and (f) analyzing the second SAM by SAMDI after removing the fraction of the second protein that is not bound to the second SAM and first protein that is not bound to the fraction of the second protein bound to the second SAM.

13. The method of claim 1 for detecting an antigen in a sample using a first antibody bound to a self-assembled monolayer (SAM), the method comprising the steps of: (a) contacting a SAM including a receptor protein with the first antibody in a manner effective to bind the first antibody to the receptor protein to form a bound first antibody; (b) contacting the bound first antibody with the sample in a manner effective to bind a first antigen in the sample to the first antibody; (c) ionizing and ablating the SAM, the bound first antibody and any of the first antigen bound to the bound first antibody and obtaining a mass spectrum including at least one mass-to-charge peak characteristic of the bound first antibody; and (d) analyzing the mass spectrum to determine if the first antigen attached to the bound first antibody.

14. The method of claim 13, wherein the binding of the antigen to the bound first antibody is analyzed without SPR or detecting a fluorescent reporter group, a radioactive reporter group or a heterologous tag that is adapted to bind a fluorescent reporter.

15. The method of claim 13, wherein the method is further characterized by one or more of the following: (a) the SAM comprises an oligo(ethylene glycol) alkanethiol adapted to selectively bind the receptor protein; (b) the SAM molecule includes a surface-bound alkanethiol moiety, an ethylene glycol moiety attached to the alkanethiol distal to the surface, a capture ligand bound to the ethylene glycol moiety and adapted to bind the receptor protein, and the receptor protein attached to the capture ligand; (c) the capture ligand includes triazacyclononane (aza); (d) the receptor protein comprises an Fc binding protein; and (e) the SAM is attached to a biochip.

16. The method of claim 13, further comprising the steps of (a) contacting the SAM with a second antibody in a manner effective to bind the second antibody to the receptor protein to form a bound second antibody and (b) contacting the bound second antibody with the sample in a manner effective to bind a second antigen in the sample to the second antibody, these steps performed prior to ionizing and ablating the SAM.

17. The method of claim 16, wherein step (a) includes contacting the SAM with a solution comprising the first antibody and the second antibody and the mass spectrum includes at least one mass-to-charge peak characteristic of the second antibody bound to the SAM.

18. The method of claim 13, wherein the first antigen is an endogenous antigen, and the method further comprises repeating steps (a)-(d) with a second SAM to obtain a second mass spectrum using a control antigen and analyzing the second mass spectrum to determine the concentration of the first antigen in the sample, the control antigen comprising a portion of the first antigen.

19. The method of claim 13, wherein the method is further characterized by one or more of the following: (a) the first antigen is full length cystatin C (CysC) or a truncated portion of CysC; (b) the capture ligand includes triazacyclononane (aza) and the receptor protein comprises Protein A, Protein G or a combination thereof; (c) the first antibody is an antibody against a protein selected from the group consisting of cystatin C (CysC), hemoglobin (hem), human albumen serum (HSA) and transferrin; (d) the first antigen is hemoglobin A and the first antibody is an antibody against hemoglobin adapted to bind to at least one of the alpha or beta portions of hemoglobin A and (e) the mass spectrum includes at least a first peak characteristic of the alpha subunit of hemoglobin A or a second peak characteristic of the beta subunit of hemoglobin A.

20.-24. (canceled)

25. A method of diagnosing a medical condition characterized by post translational modification of an antigen by using an antibody for the antigen bound to a self-assembled monolayer (SAM), the method comprising the steps of: (a) contacting a SAM including a receptor protein with the antibody in a manner effective to bind the antibody to the receptor protein to form a bound antibody; (b) contacting the bound antibody with the sample in a manner effective to bind an unmodified antigen or modified antigen in the sample to the bound antibody; (c) ionizing and ablating the SAM, the bound antibody and any of the antigen bound to the bound antibody and obtaining a mass spectrum including mass-to-charge peaks characteristic of the bound antibody the unmodified antigen, and any modified antigen; (d) analyzing the mass spectrum to determine the ratio of the modified and unmodified antigen attached to the bound antibody; and (e) correlating the ratio of the modified and unmodified antigen to diagnose the medical condition.

26. The method of claim 25, wherein the modification of the antigen includes at least one modification selected from the group consisting of: Acetylation, ADP-ribosylation, Amidation, Beta-methylthiolation, Biotinylation, Bromination, Carboxylation, Citrullination, C-Mannosylation, Cysteinylation, Deamination, Diacylglycerol modification, Dimethylation, FAD modification, Farnesylation, Hydroxyfarnesylation, FMN conjugation, Formylation, Geranyl-geranylation, Glycosylation, Glutathionylation, Hydroxylation, Lipidation, Lipoic acid modification, Methylation, Myristolation, S-nitrosylation, palmitoylation, phosphorylation, Pyruvic acid modification, Stearoylation, Sulfation, SUMOylation, Trimethylation, Ubiquitination and proteolytic cleavage.

27. The method of claim 26 for diagnosing multiple sclerosis using an antibody to cystatin C (cysC) bound to a self-assembled monolayer (SAM), the method comprising the steps of: (a) contacting a SAM including a receptor protein with the cysC antibody in a manner effective to bind the cysC antibody to the receptor protein to form a bound cysC antibody; (b) contacting the bound cysC antibody with the sample in a manner effective to bind a cysC antigen in the sample to the cysC antibody; (c) ionizing and ablating the SAM, the bound cysC antibody and any of the cysC antigen bound to the bound cysC antibody and obtaining a mass spectrum including mass-to-charge peaks characteristic of the bound cysC antibody the uncleaved cysC antigen, and any cleaved cysC antigen; and (d) analyzing the mass spectrum to determine the ratio of the uncleaved and cleaved cysC antigen attached to the bound cysC antibody.

28. The method of claim 26 for diagnosing thalassemia using an antibody to hemoglobin A (hem) bound to a self-assembled monolayer (SAM), the method comprising the steps of: (a) contacting a SAM including a receptor protein with the hem antibody in a manner effective to bind the hem antibody to the receptor protein to form a bound hem antibody; (b) contacting the bound hem antibody with the sample in a manner effective to bind a hem antigen in the sample to the hem antibody; (c) ionizing and ablating the SAM, the bound hem antibody and any of the hem antigen bound to the bound hem antibody and obtaining a mass spectrum including mass-to-charge peaks characteristic of the bound hem antibody, the alpha beta subunits of the hem antigen attached to the bound hem antibody, and beta beta subunits of the hem antigen attached to the bound hem antibody; and (d) analyzing the mass spectrum to determine the ratio of alpha and beta subunits of the hem antigen bound to the bound hem antibody.
